SEQUENCE LISTING (12 sequences)

SEQ ID NO: 1 - DNA, 660 bp, Homo sapiens
gcggcatcgggttacacagtatctagctggcaaccaggatctagttccaattccctgcttggaattattttccagagcagttccaaatcatccccttcctaggatcacaaaaagcacctacctacagtgcattccgtgctaattgggaaaatatgtctccttcctccaaggcagaggcaaccctttaggcaggtcccagagataggttcggagaccgaacagatggcctgtaaacctgaggcagaggtcaggcagccggaagggaggggctttctagggtctgtgtgtgcgtttggggagactgaaggctgcaggtggaggattggctgggggcttgtctgttggttcctctcaccccagttgatgggagtgtgggcaaatttcagccagcaagaggagaaggggtcaaagtgtgaactttctccactgcttggtcctagggggcctcaacctgcaccgcggcacaggacggccgccgtggctgtccggggttcccccctgcgcatttatgcctccatcaccctcacctctcggccacggacagcacccaggcggtggtcagccagaggccaagcgtgaggccgagcagcaggcggccagggtgcgtgttcatgtaaagcttggccacgaaccagtggcggaagcggacttgattgagag

SEQ ID NO: 2 - DNA, 1008 bp, Homo sapiens
gcctgggcggcggcggcggcggcgtgatggctccggccgcggaccgagagggctactggggccccacgacctccacgctggactggtgcgaggagaactactccgtgacctggtacatcgccgagttctgtgagtgtggcctgaggaggggagtgggggcgagagggcaccgggctgaggagacgccgtgtgaggaaggcaaagagcgaacctggccgcgaagggaggtgccaggcctggccccgggagctggaatgcggcgccctgggccagcgggaggctgagaggagcgggccgggagtccagtgtgtagagggaggagtaccggggtctgggagggaggaagggggcctgaggattgggggggcagaagagcagtgggaagtggggagcccctgctggacctaagggggaaagcctgaagagccgggttgggaatgggaattcctgcccgagagcggagtggggccaggctgggagagtggaggaccctgccccttggaatgagggcccaggacacctgctctgctgttgccaccaccagaagggtacagttcctagcttcgtctttcccccaatccgtgagaattctaccgtctttcccttcccttttcactggaatattagacctccttgctcacctccagggaacagtttcactagtctgagatctgaaccatcccacccctatcccccaggatgtcttcaagtaccagaggtcatctgctctctgagtatgattattcaactgtcatcttgcaccaggagtcgaaggcatcttgcacctagcctgtaccttctgcccctgccaggctcccaagagcacagaggaccaagtccctgctccattctgtcctatccaactatctaggagttaggggtcatctgaggacactacttccaccgactgcaccttctgaggatttaagcattcttctttagcggctgctctgtcaggcactgctggtcaggttgggcttgttctgtgtgcctatgtgggtgtctgtc

SEQ ID NO: 3 - DNA, 891 bp, Homo sapiens
gcctgcggcaaggccttcggccagagctcccagctcatccagcaccagcgggtgcactaccgcgagtagccgggcgggggctcggggctcggcctcctccctgcccccaacccaccctccaccccgtcccccacggtgggcactgcccagcaccgcatgccacgtgtccggaataaattctttttgattgttggaagtgggagccggcacctgcctgggtgagaccttggggcagcttcctatccccgaggacccgctgcgggatgggggtgatggggctgctccaccaagacctgccatacagggccacggggtccctggggtctggcgggcggcccgagtgtcgtaggggaggatctgaggcctggaggtgtcctgacttgcccaaagctgataccccaccatcaacacgggaggcgggggggggcgcgcccagagcaggggtcgaggacggggccagtctagaagtgctcacaggcctggccaggctgcctgtctgccacctgggtgaggggtctctggcaactcggttcccttatgtatttgggaggcctctgcttctgtaaatgcagcaggcttccccacgtgccctgtcagctctgctgcctccattcagtggggggcctgctgggcagcagtggcccgggcttcctctgcaccagccccttgccctggggtgtgggggcccagggtgttcaggtcttgacaggtgtgggctggtacggctgggcctgccgggccctcttcagagctgccgggacactgcttctgggcaggggagtctgggccacgaagctctgggagagctcagctgggggtggctccaagtgctgagtgccagtgattctgccagtgccttctccctgccctgcctgtgccctccgggacagc

SEQ ID NO: 4 - DNA, 758 bp, Homo sapiens
gccttcccctaggccaattctataatcaggaaagagaaagggcttttcgttgccgtgggtgagctgatgctggaggagcacagagcgatccaggaagctctctccgcagtgggagcagatgtaggtcttggaggacagcagcccccgcctctggctgaagccctcctgaccctccggcggcttaaggggctgtccgggggcctccgctctgccctccgcagccccggggtaggaattccctctgaaagggagccttggggatcgtaactgaggaggtttgggggctgcgtgtgcgatgaggccgtctgcatttttgtaggggttttctccgatgtggatcctctgatgttgcatgaagatgccctcgtcgttgaagccctttccgcacacgagacacttgtgcggcttggctcccggcggcggggtcagcagggaggggtccccgagccccagcaggctgtcgccctgggccctacgcgctggggtcttccccctctcatggatcacccggtgctgctccagctcgtgggcttccaggaaggccttcccgcagtcggagcacacgaacaggttctcgtccatgtgcgtgcggacgtgggtaataaggttggagctctggctgaagctcttgccgcactcggggcacttgtagggcttctcgccggtgtgcgtgcggcggtgctggataaggtgggagctgcggatgaagctcttgccgcagtcggaacacttgtagggcttctcaccggtgtggatgc

SEQ ID NO: 5 - DNA, 1194 bp, Homo sapiens
gcctgcaccgccaagggcgtcatgctgtggggctggcgggagcatgtctgcagtgagtgccgtccccgtgggctgcatcctggggatggggtccgggctttgagctcctgggacggggctgggggccctgagcacgggtggtccagggagaggggtcggccccctgcagccacggaccaggctccagcttcgtcagccggtggtagcaggaaaccagcaactcctatagcaaggggcggccacgtagcaggggcagaacctggggtgggcctggagctgtggcggccgagtgtgggagtgggtcccagagtgtgcactccctggccccctggccaccctggggatgggagctgggcgtctggctcttcccgtccctcacaccaccccgtggtcctctgcagacaaggatgtgggctcctgccccaactcgcaggtcttcctgtacaacctgaccacctgccagcagacctgccgctccctctccgaggccgacagccactgtctcgagggctttgcgcctgtggacggctgcggctgccctgaccacaccttcctggacgagaagggccgctgcgtacccctggccaagtgctcctgttaccaccgcggtctctacctggaggcgggggatgtggtcgtcaggcaggaagaacgatggtgggtacctgctcgggggtcaggtgtggcgtgggggcgggggagctccttctgaacctgccccaagcggagacctgggagtctctacctggggaagctgagacacccaaggctgaggggtgcctggggtggggggcgctgagaggcatcaggctcacatctgcggggaagctgcgggctgtctgtggccgtcctgcatgggccccgctcatccctggccttttccacagtgtgtgccgggatgggcggctgcactgtaggcagatccggctgatcggccagagtaagtggcactgccccggccacccctccccagccacccctccctgcctgccctggccaccctccccggccacccctcccgggcctgcctgagacccccagcttcagctggagctgaggtggcccctccgtcccacaggctgcacggccccaaagatccacatggactgcagcaacctgactgcactggccacctcgaagccccgagccctcagctgccagacgctggccgccggctatgtgcgtgttgggggc

SEQ ID NO: 6 - DNA, 1310 bp, Homo sapiens
gcaatcatagctcactgtaagcttgagctcctgggctcaagtgatcctcctacctcagactcccaaatagatggcagttaattaaaaaaacaaaattgtagagaaggggtcttgctatgttgcccaggctggtctcgaactcctgggctcaagccattctcccacctcagcctcctgagtagctgggactacaggtgcacaccactgcacccagatacgttttcttcttttttgatgaaacaagatcttgctctgttgctggggctggtctcaaacccctgggctcacgtgatcctcccgccttagcttcctaaagctctgggattacgagcgtgagctgcctcacccggccactggtgggttgctttttgttggtcttgctccccttatggaggaagaggggacggtgagagggtacgggataagcaggcatcctggcaaccagagtggcccgaggaactttctgtggaggaaatttagtgaatcaggggctccgggctggctccagagtggggcttccaccagctggtgattcttcctggaggatgaggctcaggccagggaaaggatgagcaaagcatagagtggggtgtgtgtgcgaggcagccaccggatgcccgaggcatagagtggggagtgcgtgcgaggcagccaccagacgcccgaggcatagagtggggtgtgtgtgcgaggcagccaccggacgcctgaggcatagagtggggtgtgcgtgcgaggcagccaccggacgcccgaggcatagagtggggagtgcgtgcgaggcagccaccggacgcctgaggcatagagtggggtgtgcgtgcgaggcagccactggatgcctgtgctccatgagtggctgcgctggcacagcaggactggcgcccatgggatgccacccacgtcacactgtcgtccctgtgtattcttcaatccctctacgacaggggtccccacctccggccgtggacgggtagcagcagtccctggcctgttaggaaatgggccacaaagcaggaggtaagtggcaggctagggagcattcccgccagagctcaagctcctgccggatcagtggtggcattagattctcacaggagtgtgatcctgttgtgaactgtgcgtgtgggggatttaggatgcatggtccttatgagaatctaacgcctgatgatctgaggtggtggaagtttcatcccgaaaccattctcctgcgtcccccacccctgtccacagaaaaaaccatcttccacgaaaccggtccctggtgccaaaaaggtcgggaccgattgccgctctacaacaaatgc

SEQ ID NO: 7 - DNA, 605 bp, Homo sapiens
gccctctggtggctgctgtggggaggagactgtggtggatgagggcgggagctggtgagcaggacagaggggactgcgttagtgatgagattccaagatgcccgggagaagtggcagggacgaggcggcagtgagtgtcggcacagaccccaggaggccgacagcggcttccggtcagggggcctggggaggggtcccagagcagcccgctggccacacttacccagttcggcatccgtaaggggcaggtggttgtccgcccactcctccgacttccccagcaccgtgtcgaccccactcaacaccatctggcccaagcgggagcccatgaccgattggacgccgccggtcactacggactttgtcttgtccacgccgctctgcacagcaccgcgggtcgcgtccaccgcctccgacaattgggtggccaccgtgtccttggcgctagacaccatctcttgggcccccgacaccttagacgacacaagctccttggtgtccgccaggacctaggagatgcaacagcatcagcatctctgccttccctccatatctgggcacccctcccctgcaccccaacttccagggagaccgaggcggggagc

SEQ ID NO: 8 - DNA, 1163 bp, Homo sapiens
gccgggcatgctcggcggtgtgacggctcaggactgcatttcccagaggctgcagctatccggccaatgtagcctgaaactacatttctcagcggccactggaacgacctcaatctctgcctcctcgccagttcattgtggtcgttgacccggcagcgagctttggagttcatcgagggagaagtcagcgcccagctccgaggttggagcagccccgccgggcaacttgaatttctgcaaacgaacacagcaccgggagctctgcagacctgtgtcggcgcggaacccggactgagacatgcgtgagcgttgggtggaccgggcgaggatcccgggccggcgagtgcgggagcggcagggcagggagggtgcgtcggccggggccggtgtgcatccgcgaagactgggtgcatggcctccatgcgaacctgagctattaatatttgttactattttggataaaatcactgtaattgatttatgtaaaggagcaaaagactcttcaactctcagtttaaaaaggaaacgatagttatgataccttttgcatgcagcgggaagaaatgggattgccaggaagcctcttcttgtttggaaaaaactgtataaagtatttacaccttttaaagatgagagcaatgtcatctgaaaattatcagtgcagggaaaaggacttcaaaggatctgttgtgcagattacttaactaatgacaaaattatgtaagaaaggagagcaagatgacagctgtaaacatttccatcaatctccatattgcacagaaataggacccagctttttcttaaggttcttcaattttgcattatcccacagcagtagctctctctctctagctgctaggggacagaggaaattgaaatgtcagagaatcttttctgttggttttttatttgtttgtttttaaagaagagttgctcttaattttttagttagaattaaaagaaagcatgccagagaaacttacgttttaagtaaaaagtggaaacaggtcgggtgccatggctcatgcctgtaatcccagcactttgggaagctgaggcgggtggattgcctgagctcaggagttcgagaccaccaacatggcaacatggtgaaaccccgtctctactaaaaatataaaaattagccaggcatggtggcgtgc

SEQ ID NO: 9 - DNA, 1012 bp, Homo sapiens
gctgggtgcgaatcacaccggacgtgggctcgatgtagaagtccccatcgccgtcgtccccaccctggaaggtgtacagcagacgcccattgggacctgagtcccggtccgtggcagagacctggaggatgctggtcgagggtggagcatcctcaaagatggaaccctggtagaaatcccacaggaactggggtgcattgtcattggcatcgaggatgaggatctctagggtggtggtgtctgatttctgcgggatgccgttgtcctgggccatgatggtcagcgtgtaggcgacctggttctcatagtccagctccatcatggtgtacatggtgccactgtcggggtcaatgcggaactgcggcacggggtcctgaatcacgtaggtgatgcgggcattctctcctgtgtcctcatcgttggcactgagggtagcaatggaggtgcccacaggcctgtcctcactgacactcactgtgtaatgggagctctgaaagacaggcctgtgggtgttggcatcagtgacgttgattaggacatgcgcagtgtgcgaccgtgtgccgtcggatgctgtcaccgccagcacgtactgctgctcctgcttgtagtccagaggtagcgccagggtgatgaggccgccccctctctggctgctgagtgcaaagcggttccgggtgttgccgcctgtgagctggtaggtaatcacactgttggcgtcacggtcgcgggcctgcagggtcagcacgctgctccccacggccgcatcctcattcagacgaagctcgtaggtgggctgcgtgaacaccgggtcgttgtcattcacgtccagcaccgtgatggacacgctggtggaggagctcatggggggccagccgtggtccaccgcctccaccccgaagctgtagtgctccacctcctcgcggtccagctcggcacacactgtgatccaaccggagctgttgtggatctggaaggggaagtcaggggtgggggcaggattcttaggcccagc

SEQ ID NO: 10 - DNA, 1529 bp, Homo Sapiens
gcgttttcaccgccctgtgctggaaaggcacttaggaagataatgaatataaactcacactatctggacacagatggagaaggcggtggagcattcgagtggatgattaaagagaaaaacaaatcaggaggtaaaattactgtttatgggccagggaggccacgtcctaaagtttagtggaattgtgctttagaaagaatgctgtaagaaatccagaagctgtgaagacggtaaagacaatgatgacagtgagctttcttgtttctttgaggcttccgaatgctcctccccagtctgcgtcctgctttgactggacgttgcaaacaaaagattcttgctttgtctgtctccatcctttcgaccacctccagaagctacaggaaataaacgctctttccatcctggtccctttgccacccacaaatacagagaagttgcgtctaggtaaatattaatctctgcttctgcttttccttcctgtgtgctgtgaatacaggccctgtctgcagttttacttttggctgaagtagcccatgctctagggtccatccaggaaacacacagcgcacagtcaaaccgcagacggcctgtacccacagtcaaaccacagacggcctgtatgcacaaccaaaccgcagacggcctgtacccacagtcagaccgcagacggcctgtatgaacagtcaaaccacagacggcctgtacccacagtcaaaccgcagacggcctgtacccacagtcaaaccgcagacggcctgtacccacagtcagaccgcagatggcctgtatgaacagacaaaccacaaatggcctgtattcacaatgcaaaggaaggaaaagcaaaagcaaaagttaatattcacctagatgcaacttctctgtattagtaggtggccaagggaccaggatgaaaagagcatttatttcctgtagcttctggaggtggccaggaggatggagacagacaaagcaaccagaaacacaactttccccccagccaccatccagaaacacagcttccctccagccactgtacagaaacacagcttgccccacccagccaccatccggaaacacaactcccacccacccaccatccctccaggaagccgctgtttttaatcccctcccatgagttatgaattgtgtctggtgtggtggaccctggagcatgggcttgttggctgcggttccactcgcccagcgtggggcctgggagacctggctgagctggtgtgtggtgtcctctgtacatgactccactgtggtctcccgtcctgtgggtgtgcatgcttcatccatccattgcaacgtcaacagacccctctcctccttccacttctctcctcctgttttctagtttgaaactcttaccaataatgctgctgtaaacatcttctgcatatttttggtgaatctatggatgtattcttttttttattatactttaagttttagggtacatgtgcacaatgtgcaggttagttacatatgtatacatgtgccatgctggtgtgctgcacccatta

SEQ ID NO: 11 - DNA, 1707 bp, Homo Sapiens
taatagttaatgctagcaaacagtgaaatgtaattagggcagagagacgctgaggctcattagaaagaacaacaacgctgagctgtgagccggaggaggcagccgggttctgatggaagctgcctcgaccaccagaacaacaccgcaagcgtccagcagcagtaaggggcacaagctgcctcgaccaccaggccaatgccaccagcgtccagcagcagcgaggggcacaagctgcctcgaccaccagaacaatgccgccagcgtccagcagcagtgaggggcacaagctgcctcgaccaccagaacaacgccgccagcgtccagcagcagtgaggggcacaagctgcctcgaccaccagaacaatgccgccagcgtccagcagcaacgaggggcacaagctgcctcgaccaccagaacaacaccgcaagcgtccagcagcagcgaggggcacggagagcaggcagtgcagaagtcaaacccctaacagccacaggaaactcagggcaaacggaagcgtttccattctccagcccttctttcaatattcttaacatgagcaatccatgagccctcattttgcagcccacagaacctcagccagcgtgtgaggaagaagctccaggcggcggcagccagcgtgtgaggaagaagctccacgcggcggccagtgtgtgaggaagaagctccacgcggcggcggccagtgtgtaagaaagaagctccacgcagcggccagcgtgtgaggaagaagctccacgcagcggccagcgtgtgaggaagaagctccaggcggcggcggccagcgtgtgaggaagaagctccacgcggcggccagtgtgtgaggaagaagctccacgcggcggccagtgtgtgagaaaagctccacgcagcggccagcgtgtgaggaagaagctccaggcggcggccgccagcgtgtgaggaagaagctccacgcggcgcttgctcagggaatcctgctccagggcgtgctcacttgctgttattgtgttttatttttcctgagactgtaaatggagcggatagaagttcagaaccatcggtccctcttcttcctgggtcatcctgagctcggcagtgagagcacctacgactagggagcggccgagcagagggaacaaggccgtgcccgctaaggttctcccgggacggtggcgagcccacgcttgccaggcatgacgcctcgacctccagcgtccagagcgtcccttcattggttcacaggaacttttcacatgtgtccgtccacttttcttaggaatatttatttaggtgaggttattcattctgacactggaagaaaagtgcaaaacctcgtgtggacttcgtaggtggagcatttgagttatcatcggaaaactagagcccggactgtatgaggaaggtaattcatgtttacaactgattattgctttgggtgattttctctaatgcaataataaaaatagtagaaagaaacttttcaactgtgaaacccaaacttaatattactatatcattattatcagtctttaaacacctatttcagacaagttttttaaaatataaagacaagacctaataagaggtgtgagttttacaaatataccagaaaagtgtgtgcctgaataagtgttgacccctcagagtgacccctgctggtcgcagggaacctgttcccatcacgtccccactcacccacaaggcagc

SEQ ID NO: 12 - DNA, 1606 bp, Homo Sapiens
cgaaatgcctgccaacttctgactggcaggcagtctggcaaatcaaatcgcgacctttgaaagcaaaacactgcagcatcttggcagctctgaattgggaagggatgaaggaggctgtgcctccgggttgcacgaagagtccgagtcatttctcagaaggttttgataggtgggccttagaggagacgccgccggtgagtagtgattaacaccgggaggaaggggaattgaatttaaccttcgtttttttctggaaaaagcgaagtcacctaacgtcccctagtgtacatacccttccttcttactgtcaccagcctcgccaacctgggtcccgttgccttggaatgttctttccagttttgcatcgaggccaagaggagcgggggcatgggctaccttactaaaggtgatgccaggctctaccaaaccaggaagtgacatggagttaactttgccagaatttctcctcttcgtgccgagcggctcgggcttcctggcggcagcagatggtggagttagcaggtgggatgaggggaggcgttcttggtctaagcccgcttctggaacagaggtgctgtctcctcgagttgtaagtttccagctcagtgggacgggacggaagaatgtaaccttctcgtgagccaaagccgaggaacgggaagcttggcagggaactggcgctcacctccagaagccagatcgtcgggtggtgggaaagagcgtgttttattgatttgttcagaaagaggcaaattcgaatacagacgctatgagccacggctgtctcatttgtaaaacgtgctctgtgggattggtgaaatccgtcatcgagataaacggggtgggaatggaagcagagcacctagtgaattctcattccttccttgggtcagtgaccacgtgctctaattgtggggtggttgacaacgcagaggtgactgcttgcctctcgggcatatgtaggtcctgaagaaatgcttcgaaatcaggaaaagagagtcaccaggtgaaaagtatgtgtcttataagggagtacagcttgcaaagggttcctccaggctttcagtccggactcccacccagctgagggagagccttcaatctttgcaggcgatgcttcagaggcctgcgagttcctgaggcagagagggaagctgctttttaaaagaaaaattaaaccacagcaatgccaaccaccacaaacaaaagcaaaaccagaaaagcacttgggcaaactactctgaaggatgttaggaggcctgggttcccaccactcccttgattgacatgtcgctaaggtctgttggctttctctgacctcctgtgggtggggctgagtatatctgcttgttgaagctctggagttgtggttgattaggccttagaaaggcattcttgactgcgaaggggccacagtgcacccagtgcttaaccagcctgcatttagtcagtcggttggtatgtatacaagtcactgtcaggctctgggctagatctcatttagtcagttggttggtatgtatagaagtcactgtcaggctctgggctagatctcagctgggagcaactgaacagggtattccgcaaacacctcactggagtttgc

(19) United States
(12) Patent Application Publication
(10) Pub. No.: US 2016/0208334 A1
(43) Pub. Date: Jul. 21, 2016

(54) BIOMARKERS FOR DETECTION OF COLORECTAL CANCER

(71) Applicant: US BIOMARKERS, INC., Buffalo Grove, IL (US)

(72) Inventors: Victor V. Levenson, Buffalo Grove, IL (US); Anatoliy A. Melnikov, Buffalo Grove, IL (US)

(73) Assignee: US BIOMARKERS, Inc., Buffalo Grove, IL (US)

(21) Appl. No.: 14/915,419

(22) PCT Filed: Sep. 19, 2014
(86) PCT No.: PCT/US2014/056602

§ 371 Date: Feb. 29, 2016

Related U.S. Application Data
(60) Provisional application No. 61/884,374, filed on Sep. 30, 2013.

(51) Int. Cl. C12Q 1/68

(52) U.S. Cl. CPC C12Q 1/6886; C12Q 2600/154; C12Q 2600/112; C12Q 2600/158

(57) ABSTRACT

Provided are kits comprising biomarkers for detection of early stages (I and II) of colorectal cancer and methods for diagnosis and treatment of colorectal cancer at early stages (I and II).

### BACKGROUND OF THE INVENTION

1. Field of the Invention

The invention disclosed herein relates generally to the fields of clinical testing in oncology. Particularly, the invention provides a biomarker comprising abnormally methylated DNA fragments in a sample for detection of early stages (I and II) of colorectal cancer. More particularly, the invention provides methods for detection of early stages (I and II) of colorectal cancer using a marker comprising abnormally methylated DNA fragments in a sample.

2. Description of Related Art

Colorectal cancer (CRC), is the third most common form of cancer and the second leading cause of death among cancers worldwide, with approximately 1,000,000 new cases of CRC and 50, 000 deaths related to CRC each year (Bandres E, Zarate R, Ramirez N, Abajo A, Bitarte N, Garcia-Foncillas J: Pharmacogenomics in colorectal cancer: the first step for individualized-therapy, World J Gastroenterol 2007, 13(44):5888-5901; Kim H-J, Yu M-H, Kim H, Byun J, Lee CH: Non-invasive molecular biomarkers for the detection of colorectal cancer, BMB Rep 2008, 41(10):685-692).

Most colorectal cancers develop slowly, beginning as small benign colorectal adenomas which progress over several decades to larger and more dysplastic lesions which eventually become malignant. This gradual progression provides multiple opportunities for prevention and intervention.

The currently used methods for the early detection of CRC are the Faecal Occult Blood Test (FOBT) and the endoscopy. FOBT is simple, inexpensive and the least invasive method of screening available. Also, it has been shown through prospective randomized trials that FOBT reduces CRC mortality, and consequently the evidence for its use is robust.

However, FOBT presents relatively high false negative and false positive rates, and it has particularly poor sensitivity for the detection of early-stage lesions (see e.g., Burch J A, Soares-Weiser K, St John D J, Duffy S, Smith S, Kleijnen J, Westwood M: Diagnostic accuracy of faecal occult blood tests used in screening for colorectal cancer: a systematic review, J Med Screen 2007, 14(3):132-137; Allison J E, Tekawa I S, Ransom L J, Adrain A L: A comparison of fecal occult blood tests for colorectal-cancer screening, N Engl J Med 1996, 334(3):155-159; Greenberg P D, Bertario L, Gnauck R, Kronborg O, Hardcastle J D, Epstein M S, Sadowski D, Sudduth R, Zuckerman G R, Rockey D C: A prospective multicenter evaluation of new fecal occult blood tests in patients undergoing colonoscopy, Am J Gastroenterol 2000, 95(5):1331-1338).

In an attempt to improve on the false positive rates of FOBT, a new Faecal Immunochemical testing (FIT) has been developed. It has slightly superior performance characteristics but at a greatly increased financial cost, and its implementation has not been effective as yet (see Newton K F, Newman W, Hill J: Review of biomarkers in colorectal cancer, Colorectal Disease 2012, 14(1):3-17).

On the other hand, colonoscopy offers significant improvements in detection rates for CRC but it also has important disadvantages associated, such as inconvenience, high economic burden and potential major complications (bleeding, perforation) (see e.g., Winawer S, Fletcher R, Rex D, Bond J, Burt R W, Ferrucci J, Ganiats T, Levin T, Woolf S, Johnson D, et al.: Colorectal cancer screening and surveillance: clinical guidelines and rationale-Update based on new evidence, Gastroenterol 2003, 124:544-560; Greenen J E, Schmitt M G, Wu W C, Hogan W J: Major complications of colonoscopy: bleeding and perforation, Am J Dig Dis 1975, 20:231-235).

Since none of the currently available methods are optimal, there is an urgent necessity of new diagnostic approaches in order to improve the outcome of CRC screening programs.

DNA methylation biomarkers for noninvasive diagnosis of CRC and precursor lesions have been extensively studied. Different panels have been reported attempting to improve current protocols in clinical practice and several biomarkers (for example SEPT9 test) have been established to date (see e.g., Lofton-Day C. et al., DNA methylation biomarkers for blood-based colorectal cancer screening, Clin Chem. 2008 February, 54(2):414-23; Grützmann R. et al., Sensitive detection of colorectal cancer in peripheral blood by septin 9 DNA methylation assay, PLoS One. 2008, 3(11):e3759; deVos T. et al., Circulating methylated SEPT9 DNA in plasma is a biomarker for colorectal cancer, Clin Chem. 2009 July, 55(7):1337-46; Ahlquist D. A. et al., The stool DNA test is more accurate than the plasma septin 9 test in detecting colorectal neoplasia, Clin Gastroenterol Hepatol. 2012 March, 10(3):272-7.e1; Ladabaum U. et al., Colorectal Cancer Screening with Blood-Based Biomarkers: Cost-Effectiveness of Methylated Septin 9 DNA versus Current Strategies, Cancer Epidemiol Biomarkers Prey. 2013 September, 22(9):1567-76). However, these tests suffer from low sensitivity in detecting early stages (I and II) of colorectal cancer and no definite biomarkers have been identified to date that can be reliably used for detecting CRC in blood samples. Thus, there is a clinical need for identifying specific biomarkers for early detection of CRC that can be tested in a noninvasive manner.

### SUMMARY OF THE INVENTION

It is against the above background that the present invention provides certain advantages and advancements over the prior art.

Although this invention is not limited to specific advantages or functionality, it is noted that the invention disclosed herein provides methods for detecting cancer, comprising:

(a) analyzing a biological sample from a subject to detect a presence of a biomarker in the sample, comprising
i. obtaining a DNA sample from a subject;
ii. digesting the DNA sample with a methylation-sensitive restriction enzyme in the presence of a glycol compound;
iii. amplifying the digested sample of step (ii);
iv. quantifying amplification results from step (iii) using a real-time quantitative PCR; and
v. analyzing DNA methylation status within a recognition site of the methylation-sensitive restriction enzyme to detect a presence of the biomarker in the sample;

(b) determining a methylation status of the biomarker detected in step (v); and

(c) comparing the methylation status of the biomarker detected in the sample to cancer-positive and/or cancer-negative reference methylation status of the biomarker to detect whether the subject has cancer.

In preferred embodiments, the step of comparing further comprises:

(a) determining probabilities (P) of the biomarker being methylated or unmethylated;

(b) determining cumulative probabilities of error (ρ) associated with probabilities (P) of the biomarker being methylated or unmethylated;

(c) determining cumulative probabilities of error for the biomarker in a healthy and a diseased state; and

(d) detecting that the subject has cancer if the cumulative probabilities of error for the biomarker in the healthy state is more than the cumulative probabilities of error for the biomarker in the diseased state.

In preferred embodiment, the subject is a mammal, wherein the mammal is a human.

In one aspect, the sample is a biological sample comprising blood, blood plasma, urine or saliva.

In further aspect, the biomarker comprises one or more DNA fragments of SEQ ID Nos. 1-12.

In further aspect, the cancer is colorectal cancer.

In further aspect, the colorectal cancer comprises early stage I and II colorectal cancer or late stage colorectal cancer.

In further aspect, the DNA comprises a genomic DNA.

In further aspect, the DNA sample is between about 1 pg and about 1 ng.

In further aspect, the DNA sample is about 300 pg.

In further aspect, the methylation-sensitive restriction enzyme comprises Hin6I.

In further aspect, amplifying comprises amplifying using phi29 DNA polymerase.

In further aspect, the amplifying further comprises amplifying using a single stranded DNA binding protein of E. coli.

In further aspect, the real-time quantitative PCR comprises TaqMan qPCR.

In further aspect, determining the DNA methylation status and/or probability of a methylation status comprises determining threshold cycle (CT) values.

The invention disclosed herein further provides a biomarker for detecting cancer, wherein the biomarker comprises one or more DNA fragments of SEQ ID Nos. 1-12.

In one aspect, the cancer is colorectal cancer.

In further aspect, the colorectal cancer comprises early stage I and II colorectal cancer or late stage colorectal cancer.

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows qPCR profile of methylated and unmethylated fragments.

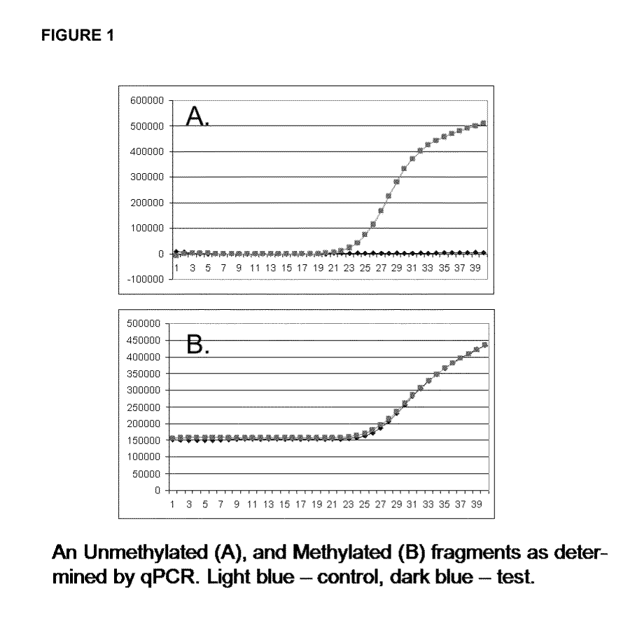

Skilled artisans will appreciate that elements in the figures are illustrated for simplicity and clarity and have not necessarily been drawn to scale. For example, the dimensions of some of the elements in the figures can be exaggerated relative to other elements to help improve understanding of the embodiment(s) of the present invention.

### DETAILED DESCRIPTION OF THE INVENTION

All publications, patents and patent applications cited herein are hereby expressly incorporated by reference for all purposes.

Methods well known to those skilled in the art can be used to construct genetic expression constructs and recombinant cells according to this invention. These methods include in vitro recombinant DNA techniques, synthetic techniques, in vivo recombination techniques, and PCR techniques. See, for example, techniques as described in Maniatis et al., 1989, MOLECULAR CLONING: A LABORATORY MANUAL, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

### Definitions

Before describing the present invention in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the terms “polynucleotide”, “nucleotide”, “oligonucleotide”, and “nucleic acid” can be used interchangeably to refer to nucleic acid comprising DNA, RNA, derivatives thereof, or combinations thereof.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

The terms “compound”, “test compound,” “agent”, and “molecule” are used herein interchangeably and are meant to include, but are not limited to, peptides, nucleic acids, carbohydrates, small organic molecules, natural product extract libraries, and any other molecules (including, but not limited to, chemicals, metals, and organometallic compounds).

The term “detection” is used herein to refer to any process of observing a marker, or a change in a marker (such as for example the change in the methylation state of the marker), in a biological sample, whether or not the marker or the change in the marker is actually detected. In other words, the act of probing a sample for a marker or a change in the marker, is a “detection” even if the marker is determined to be not present or below the level of sensitivity. Detection may be a quantitative, semi-quantitative or non-quantitative observation.

The term “including” is used herein to mean, and is used interchangeably with, the phrase “including but not limited to.”

The term “isolated” as used herein with respect to nucleic acids, such as DNA or RNA, refers to molecules in a form which does not occur in nature. Moreover, an “isolated nucleic acid” is meant to include nucleic acid fragments which are not naturally occurring as fragments and would not be found in the natural state.

The term “or” is used herein to mean, and is used interchangeably with, the term “and/or”, unless context clearly indicates otherwise.

The terms “phenotype” or “phenotypic status” are used herein interchangeably and are meant to describe whether a subject has or does not have a particular disease.

The term “healthy state” means that a subject does not have a particular disease.

The term “diseased state” means that a subject has a particular disease.

“Sample” or “biological sample” means biological material isolated from a subject. The biological sample may contain any biological material suitable for detecting the desired biomarkers, and may comprise cellular and/or non-cellular material from the subject. The sample may be isolated from any suitable biological tissue or fluid such as, for example but not limited to, blood, blood plasma, urine or saliva. A “sample” includes any material that is obtained or prepared for detection of a molecular marker or a change in a molecular marker such as for example the methylation state, or any material that is contacted with a detection reagent or detection device for the purpose of detecting a molecular marker or a change in the molecular marker.

A “subject” is any organism of interest, generally a mammalian subject, such as, for example, a human, monkey, mouse, or rabbit, and preferably a human subject.

The term “biomarker” means an organic biomolecule(s) a marker and/or a panel of DNA fragments, such as for example but not limited to a panel of methylated or unmethylated DNA fragments, which are differentially present (i.e., present with an incorrect methylation status) in a biological sample taken from a subject or a group of subjects having a first phenotype (e.g., having a disease) as compared to a biological sample from a subject or group of subjects having a second phenotype (e.g., not having the disease).

A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent).

A biomarker is preferably differentially present between different phenotypic statuses at a level that is statistically significant (i.e., a p-value less than 0.05 and/or a q-value of less than 0.10 as determined using the following, among others, tests: Welch's T-test, Wilcoxon's rank-sum Test, ANOVA, Kruskal-Wallis, Mann-Whitney, and odds ratio):

A biomarker, as described above, may provide a measure of relative risk that a subject belongs to one phenotypic status or another. Therefore, the biomarker may be useful for disease diagnostics.

A “reference methylation status” of a biomarker means a methylation status of the biomarker that is indicative of a particular disease state, phenotype, or lack thereof, as well as combinations of disease states, phenotypes, or lack thereof. A “positive” reference methylation status of the biomarker means a methylation status that is indicative of a particular disease state or phenotype. A “negative” reference methylation status of the biomarker means a methylation status that is indicative of a lack of a particular disease state or phenotype. Specifically, a reference methylation status of the biomarker in healthy subjects may be used to determine a “negative reference methylation status.”

For example, a “colorectal cancer-positive reference methylation status” of a biomarker means a methylation status the biomarker that is indicative of a positive diagnosis of colorectal cancer in a subject, and a “colorectal cancer-negative reference methylation status” of a biomarker means a methylation status of the biomarker that is indicative of a negative diagnosis of colorectal cancer in a subject.

A “reference methylation status” of a biomarker may be a combination of relative methylation statuses of one and/or several DNA fragments, and such reference methylation status may be tailored to specific populations of subjects (e.g., a reference methylation status may be age-matched so that comparisons may be made between methylation statuses of DNA fragments in samples from subjects of a certain age and for a particular disease state, phenotype, or lack thereof in a certain age group). Such reference methylation status may also be tailored to specific techniques that are used to measure methylation status in biological samples (e.g., DNA methylation, etc.), where the methylation status may differ based on the specific technique that is used.

“Abnormally methylated” as used herein with respect to DNA fragments from a sample, refers to fragments that are methylated in the sample when such fragments are supposed to be unmethylated or fragments that are unmethylated in the sample when such fragments are supposed to be methylated with respect to a “reference methylation status” discussed above.

### Overview

In certain aspects, the invention relates to a biomarker, wherein the biomarker comprises one or more abnormally methylated DNA fragments (see Table 1) from a sample from a subject for detection of early stages (I and II) of colorectal cancer and methods for the in vitro detection of early stages (I and II) of colorectal cancer by determining the presence of said biomarker in the sample from the subject.

Generally, DNA methylation in fragments of Table 1 may be determined for biological samples from subjects diagnosed with colorectal cancer as well as from one or more other groups of human subjects (e.g., healthy control subjects not diagnosed with colorectal cancer), as well as from human subjects diagnosed with early stage I and II colorectal cancer and human subjects diagnosed with late stages colorectal cancer.

### Biomarkers

Methylation status of the one or more DNA fragments comprising the biomarker in biological samples from a subject having colorectal cancer was compared to the methylation status of the one or more DNA fragments comprising the biomarker in biological samples from the one or more other groups of subjects. A biomarker comprising one or more abnormally methylated DNA fragments, including those abnormally methylated at a level that is statistically significant, in the methylation profile of samples from subjects with colorectal cancer as compared to another group (e.g., healthy control subjects not diagnosed with colorectal cancer) was used to distinguish those groups. In addition, abnormally methylated DNA fragments, including those abnormally methylated at a level that is statistically significant, in the methylation profile of samples from subjects with early stage I and II colorectal cancer as compared to late stage colorectal cancer were also identified as biomarkers to distinguish those groups.

The biomarker is discussed in more detail herein. The biomarker comprising one or more DNA fragments (see Table 1) was used for distinguishing subjects having colorectal cancer (early stage I and II and/or late stage) vs. control subjects not diagnosed with colorectal cancer. The sequence information for DNA fragments comprising the biomarker (see Table 1) is shown in Table 7. DNA methylation sites are shown in bold and are underlined.

TABLE 1
DNA fragments comprising the biomarker.

| | Status of | | | | |
|---|---|---|---|---|---|
| | methylation | | | | |
| # | Healthy | CRC | GENE | Location | Known function |
| 1 | Unmeth | Meth | KCNN4 | 19q13.2 | Ca-activated |
| | | | | | K-chanel, |
| | | | | | regulates |
| | | | | | calcium influx |
| 2 | Unmeth | Meth | ACER3 | 11q13.5 | alkaline ceramidase |
| | | | | | 3, positively |
| | | | | | regulates |
| | | | | | cell proliferation |
| 3 | Unmeth | Meth | GLI4 | 8q24.3 | GLI family zinc |
| | | | | | finger 4; glioma- |
| | | | | | assoc. oncogene |
| | | | | | family |
| 4 | Unmeth | Meth | ZNF629 | 16p11.2 | |
| 5 | Unmeth | Meth | MUC2 | 11p15.5 | Mucin 2; loss of |
| | | | | | expression - |
| | | | | | recurrence |
| 6 | Unmeth | Meth | HDAC4 | 2q37.3 | Histone deacetylase |
| | | | | | 4 promotes CRC via |
| | | | | | repression of p21 |
| 7 | Meth | Unmeth | PLIN3 | 19p13.3 | Perilipin3 binds |
| | | | | | directly to the |
| | | | | | GTPase RAB9 |
| | | | | | (RAB9A) |
| 8 | Meth | Unmeth | ZNF30 | 19q13.11 | |
| 9 | Meth | Unmeth | CELSR1 | 22q13.3 | cadherin, EGF LAG |
| | | | | | seven-pass G-type |
| | | | | | receptor 1 |
| 10 | Meth | Unmeth | unkown | chr8: | |
| | | | | 1094666- | |
| | | | | 1094715 | |
| 11 | Meth | Unmeth | unkown | chr2: | |
| | | | | 583162- | |
| | | | | 583222 | |
| 12 | Meth | Unmeth | NIPAL3 | 1p36.12- | |
| | | | | p35.1 | |

Although the identities of some of the biomarkers are not known at this time, such identities are not necessary for the identification of the biomarkers in biological samples from subjects, as the “unnamed” biomarkers have been sufficiently characterized by analytical techniques to allow such identification. The methodology for analytical characterization of all such “unnamed” biomarkers is described in Example 1.

### Detection of Colorectal Cancer

After the methylation status of the one or more DNA fragments comprising the biomarker are determined in the sample, the methylation status of the one or more DNA fragment comprising the biomarker compared to colorectal cancer-positive and/or colorectal cancer-negative reference methylation status to detect or aid in detecting whether the subject has colorectal cancer. Methylation status of the one or more DNA fragment comprising the biomarker in a sample, including those abnormally methylated or unmethylated a level that is statistically significant, matching the colorectal cancer-positive reference methylation status (e.g., methylation status that is the same as the reference methylation status, substantially the same as the reference methylation status, above and/or below the minimum and/or maximum of the reference methylation status, and/or within the range of the reference methylation status) are indicative of a detecting of colorectal cancer in the subject. Methylation status of the one or more DNA fragment comprising the biomarker in a sample, including those abnormally methylated or unmethylated a level that is statistically significant, matching the colorectal cancer-negative reference methylation status (e.g., methylation status that is the same as the reference methylation status, substantially the same as the reference methylation status, above and/or below the minimum and/or maximum of the reference methylation status, and/or within the range of the reference methylation status) are indicative of a detection of no colorectal cancer in the subject.

The methylation status of the one or more DNA fragments comprising the biomarker may be compared to colorectal cancer-positive and/or colorectal cancer-negative reference methylation status using various techniques, including but not limited to a simple comparison (e.g., a manual comparison) of the methylation statuses in the biological sample to colorectal cancer-positive and/or colorectal cancer-negative reference levels. The methylation status of the one or more DNA fragments comprising the biomarker in the biological sample may also be compared to colorectal cancer-positive and/or colorectal cancer-negative reference methylation status using one or more statistical analyses (e.g., t-test, Welch's T-test, Wilcoxon's rank sum test, random forest).

The identification of a biomarker comprising one or more DNA fragments for colorectal cancer allows for the detection of (or for aiding in the detection of) colorectal cancer in asymptomatic subjects and/or subjects presenting with one or more symptoms of colorectal cancer. A method of detecting (or aiding in detecting) whether a subject has colorectal cancer may comprise (1) analyzing a biological sample from a subject to determine the presence of a biomarker comprising one or more DNA fragments for colorectal cancer in the sample and (2) comparing the methylation status of the one or more DNA fragments comprising the biomarker in the sample to colorectal cancer-positive and/or colorectal cancer-negative reference methylation status of the one or more DNA fragments comprising the biomarker. When such a method is used to aid in the detection of colorectal cancer, the results of the method may be used along with other methods (or the results thereof) useful in the clinical determination of whether a subject has colorectal cancer.

The methods of detecting (or aiding in detecting) whether a subject has colorectal cancer may also be conducted specifically to detect (or aid in detecting) whether a subject has an early stage I and II colorectal cancer and/or late stage colorectal cancer. Such methods may comprise (1) analyzing a biological sample from a subject to determine the presence of a biomarker comprising one or more DNA fragments in the sample of early stage I and II colorectal cancer (and/or late stage colorectal cancer) in the sample, (2) determining the methylation status of the one or more DNA fragment comprising the biomarker, and (3) comparing the methylation status of the one or more DNA fragment comprising the biomarker in the sample to an early stage I and II colorectal cancer-positive and/or an early stage I and II colorectal cancer-negative reference methylation status (or late stage colorectal cancer-positive and/or late stage colorectal cancer-negative reference methylation status) in order to detect (or aid in the detection of) whether the subject has an early stage I and II colorectal cancer (or late stage colorectal cancer).

Detection of (or aiding in the detection of) colorectal cancer using above described biomarker is based on detecting abnormal methylation status of DNA fragments to detect early stages (I and II) of colorectal cancer. While each fragment is not sufficient to identify cancer with sufficient accuracy, the combination of relative probabilities of several fragments identifies the disease with very high accuracy. Abnormal methylation of the fragments (i.e., methylation that does not correspond to methylation status for the same fragments in healthy subjects) is detected using the technology for analysis of DNA methylation in ultra-small samples as described below.

Thus, detection of (or aiding in the detection of) colorectal cancer using above described biomarker is based, in part, on determining the probabilities (P) of consensus reading (in regards to methylated status) for DNA fragments comprising the biomarker as shown in Table 2. The probabilities are recorded together with the errors (ρ) ρ=1−P for each of the DNA fragments comprising the biomarker.

TABLE 2
Methylation status of DNA fragments comprising the biomarker.

| | | 1 | 2 | 3 | 4 | 5 | 6 | 7 | 8 | 9 | 10 | 11 | 12 |
|---|---|---|---|---|---|---|---|---|---|---|---|---|---|
| Healthy (consensus) | | M | M | M | M | M | M | M | M | M | M | M | M |
| Probability of consensus reading M | P | 0.2 | 0.3 | 0.3 | 0.1 | 0.3 | 0.2 | 0.7 | 0.8 | 0.7 | 0.8 | 0.8 | 0.8 |
| Error | ρ = 1 − P | 0.8 | 0.7 | 0.7 | 0.9 | 0.7 | 0.8 | 0.3 | 0.2 | 0.3 | 0.2 | 0.2 | 0.2 |
| Cancer (consensus) | | M | M | M | M | M | M | M | M | M | M | M | M |
| Probability of consensus reading M | P | 0.7 | 0.7 | 0.8 | 0.7 | 0.7 | 0.8 | 0.2 | 0.3 | 0.3 | 0.3 | 0.2 | 0.2 |
| Error | ρ = 1 − P | 0.3 | 0.3 | 0.2 | 0.3 | 0.3 | 0.2 | 0.8 | 0.7 | 0.7 | 0.7 | 0.8 | 0.8 |

The first six DNA fragments are selected to produce unmethylated readout in healthy control subjects not diagnosed with colorectal cancer and methylated status in subjects diagnosed with colorectal cancer. Accordingly, the probabilities (P) of these DNA fragments being methylated are small and the errors (ρ) are large in healthy subjects, while the probabilities (P) of these DNA fragments being methylated are large and the errors (ρ) are small in subjects diagnosed with colorectal cancer.

The last six DNA fragments are selected to produce methylated readout in healthy control subjects not diagnosed with colorectal cancer and unmethylated status in subjects diagnosed with colorectal cancer. Accordingly, the probabilities (P) of these DNA fragments being methylated are large and the errors (ρ) are small in healthy subjects, while the probabilities (P) of these DNA fragments being unmethylated are small and the errors (ρ) are large in subjects diagnosed with colorectal cancer.

The error rates associated with probabilities for each DNA fragment being either methylated or unmethylated in healthy subjects and subjects diagnosed with colorectal cancer are summarized in Table 3, which are used for determining whether a subject has colorectal cancer as discussed in Example 2.

TABLE 3
Probabilities of error for each fragment being
unmethylated or methylated in healthy subjects
and subjects diagnosed with colorectal cancer.

| | |
|---|---|
| Healthy sample | Cancer sample |
| ρ = 1 − P | ρ = 1 − P |

| | |
|---|---|
| Status | Status |

| # | M | UM | # | M | UM |
|---|---|---|---|---|---|
| 1 | 0.8 | 0.2 | 1 | 0.3 | 0.7 |
| 2 | 0.7 | 0.3 | 2 | 0.3 | 0.7 |
| 3 | 0.7 | 0.3 | 3 | 0.2 | 0.8 |
| 4 | 0.9 | 0.1 | 4 | 0.3 | 0.7 |
| 5 | 0.7 | 0.3 | 5 | 0.3 | 0.7 |
| 6 | 0.8 | 0.2 | 6 | 0.2 | 0.8 |
| 7 | 0.3 | 0.7 | 7 | 0.8 | 0.2 |
| 8 | 0.2 | 0.8 | 8 | 0.7 | 0.3 |
| 9 | 0.3 | 0.7 | 9 | 0.7 | 0.3 |
| 10 | 0.2 | 0.8 | 10 | 0.7 | 0.3 |
| 11 | 0.2 | 0.8 | 11 | 0.8 | 0.2 |
| 12 | 0.2 | 0.8 | 12 | 0.8 | 0.2 |

The methylation status of one or more DNA fragment comprising the biomarker may be determined in the methods of detecting and methods of aiding in detecting whether a subject has colorectal cancer. For example, the methylation status of one DNA fragment, two or more DNA fragments, three or more DNA fragments, four or more DNA fragments, five or more DNA fragments, six or more DNA fragments, seven or more DNA fragments, eight or more DNA fragments, nine or more DNA fragments, ten or more DNA fragments, etc., including a combination of all of the DNA fragments in Table 1, may be determined and used in such methods.

Determining methylation status of combinations of the DNA fragments may allow greater sensitivity and specificity in detecting colorectal cancer and aiding in the detection of colorectal cancer, and may allow better differentiation of colorectal cancer from other colorectal disorders (e.g., appendicitis, benign adenoma, ulcerative colitis, Crohn's disease, diverticular disease, Irritable Bowel Syndrome, etc.) or other cancers that may have similar or overlapping biomarkers to colorectal cancer (as compared to a subject not having colorectal cancer).

### Discovery of Colorectal Biomarkers

The colorectal cancer biomarkers described herein were discovered using analysis of DNA methylation in selected fragments using ultra-small samples (300 pg or less) of genomic DNA extractable from clinical samples.

Briefly, DNA samples obtained from a subject was divided into two parts; one part was treated with the methylation-sensitive restriction enzyme and/or methylation-dependent restriction enzyme in defined conditions, while the other part was incubated without the enzyme and serves as the control. Genomic DNA in both parts was amplified using genome-wide amplification with phi29 enzyme, and selected fragments were analyzed using TaqMan quantitative PCR. The ΔCt of the restriction enzyme-treated and control parts of the sample were compared to determine the methylation status and/or probability of a methylation status of the recognition sites for the restriction enzyme within the selected fragments.

Digestion of Ultra-Small Samples (300 pg or less) of Genomic DNA with a Methylation-Sensitive Restriction Enzyme

In one embodiment, methylation-sensitive restriction enzyme Hin6I (ThermoScientific) was used for restriction digestion (see Example 1). This enzyme recognizes the site GCGC, and does not cut DNA if the second nucleotide (cytosine) is methylated. Importantly, the reaction conditions generally used for restriction digest with Hin6I (see e.g., http://www.thermoscientificbio.com/search/?term=Hin6I) are not suitable for effective and efficient digestion of genomic DNA at ultra-low levels (300 pg or less).

Alternatively, a restriction digestion may be carried for example with the following, but not limiting, methylation-sensitive and methylation-dependent restriction enzymes and their isoschizomers as shown below in Tables 4 and 5.

TABLE 4
Methylation-sensitive restriction enzymes.

| Restriction Enzyme | Recognition Sequence | Catalog Number |
|---|---|---|
| Aat II | GACGT↓C | Clontech: 1112A/B |
| Acc II | CG↓CG | Clontech: 1002A/B |
| Aor13H I | T↓CCGGA | Clontech: 1224A/B |
| Aor51H I | AGC↓GCT | Clontech: 1118A/B |
| BspT104 I | TT↓CGAA | Clontech: 1225A/B |
| BssH II | G↓CGCGC | Clontech: 1119A/B |
| Cfr10 I | R↓CCGGY | Clontech: 1120A/B |
| Cla I | AT↓CGAT | Clontech: 1034A/B/AH/BH |
| Cpo I | CG↓GWCCG | Clontech: 1035A/B |
| Eco52 I | C↓GGCCG | Clontech: 1039A/B |
| Hae II | RGCGC↓Y | Clontech: 1052A/B |
| Hap II | C↓CGG | Clontech: 1053A/B/AH/BH |
| Hha I | GCG↓C | Clontech: 1056A/B |
| Mlu I | A↓CGCGT | Clontech: 1071A/B/AH/BH |
| Nae I | GCC↓GGC | Clontech: 1155A/B |
| Not I | GC↓GGCCGC | Clontech: 1166A/B/BH |
| Nru I | TCG↓CGA | Clontech: 1168A/B |
| Nsb I | TGC↓GCA | Clontech: 1226A/B |
| PmaC I | CAC↓GTG | Clontech: 1177A/B |
| Psp1406 I | AA↓CGTT | Clontech: 1108A/B |
| Pvu I | CGAT↓CG | Clontech: 1242A/B |
| Sac II | CCGC↓GG | Clontech: 1079A/B |
| Sal I | G↓TCGAC | Clontech: 1080A/B/AH/BH |
| Sma I | CCC↓GGG | Clontech: 1085A/B/AH/BH |
| SnaB I | TAC↓GTA | Clontech: 1245A/B |
| DpnII | ↓GATC | NEB: R0543S |
| HpaII | C↓CGG | NEB: R0171S |
| MspI | C↓CGG | NEB: R0106S |
| Sall-HF | G↓TCGAC | NEB: R3138S |
| ScrFI | CC↓NGG | NEB: R0110S |

Wherein N = A or C or G or T; D = A or G or T; B = C or G or T; V = A or C or G; R = A or G; S = C or G; W = A or T; Y = C or T.

See e.g., http://www.clontech.com/takara/US/Products/Epigenetics/DNA_Preparation/MSRE_Over view) and https://www.neb.com/products/epigenetics/methylation-sensitive-restriction-enzymes.

TABLE 5
Methylation-dependent restriction enzymes.

| Restriction Enzyme | Recognition Sequence | Catalog Number |
|---|---|---|
| BisI | G^{m}CNGC | |
| GlaI | G^{m}CG^{m}C | |
| FspEI | C^{m}C(N)₁₂↓ | NEB: R0662S |
| LpnPI | C^{m}CDG(N)₁₀↓ | NEB: R0663S |
| McrBC | Pu^{m}C(N₄₀₋₃₀₀₀)Pu^{m}C↓ | NEB: M0272S |
| MspJI | ^{m}CNNR(N)₁₂↓ | NEB: R0661S |
| SgeI | ^{m}5C N N G (N)₉↓ | ThermoScientific: ER2211 |
| MspJI | ^{m}CNNR(N)₉↓ | NEB: R0661S |
| FspEI | C^{m}C(N)₁₂↓ | NEB: R0662S |
| AspBHI | YSCNS(N)₈↓(N)₁₂SNGSR | |

Wherein N = A or C or G or T; D = A or G or T; B = C or G or T; V = A or C or G; R = A or G; S = C or G; W = A or T; Y = C or T.

See e.g., https://www.neb.com/products/epigenetics/methylation-dependent-restriction-enzymes; Karni et al., PNAS (2011); Murray, Microbiology (2002); Sitaraman et al., Gene (2011).

In general, the cleavage by Hin6I or any other methylation-sensitive or methylation-dependent enzyme can be detected, for example, but not limiting to, quantitative PCR (qPCR) and next generation sequencing (NGS).

In order to accelerate digestion of a genomic DNA present at an ultra-low concentration (300 pg or less), the inventors have advantageously determined the “optimal conditions” for digestion with a methylation-sensitive restriction enzyme Hin6I (see Example 1).

The efficiency with which a restriction enzyme cuts its recognition sequence at different locations in a piece of DNA can vary 10 to 50-fold. This is may be due to influences of sequences bordering the recognition site, which perhaps can either enhance or inhibit enzyme binding or activity (see, e.g., http://www.vivo.colostate.edu/hbooks/genetics/biotech/enzymes/cuteffects.html).

“Optimal conditions” of the digestion reaction are defined as “complete digestion” of all unmethylated sites GCGC by the methylation-sensitive restriction enzyme Hin6I within an acceptable (<5 hr) timeframe. “Complete digestion” is defined as the absence of a specific PCR product from a target within the genome when the target contains an unmethylated site GCGC, and 40 cycles of PCR are performed. Additionally, the “complete” digestion is defined as the absence of a specific PCR product following 40 cycles of qPCR, when the undigested part of the sample demonstrates PCR product with C_{T} range of between 17 and 27.

Surprisingly and unexpectedly, the inventors have discovered that DNAzoI Direct, a glycol compound previously used for storing and/or processing of biological samples for direct use in PCR, may be advantageously added to the restriction digestion of a genomic DNA with a methylation-sensitive restriction enzyme Hin6l. For example, as described in U.S. Pat. No. 7,727,718 (incorporated herein by reference in its entirety) DNAzoI Direct, a glycol compound, may comprise ethylene glycol, polyethylene glycols, polyglycol, propylene glycol, polypropylene glycol and glycol derivatives including polyoxyethylene lauryl ether, octylphenol-polyethylene glycol ether, and polyoxyethylene cetyl ether.

The glycol compounds of this invention may further comprise 1,2-propanediol, 1,3-butanediol, 1,4-butanediol, 1,4-cyclohexanedimethanol-, 1,6-hexanediol, butylene glycol, diethylene glycol, dipropylene glycol, ethylene and propylene glycol (including ethylene and propylene glycol monomers and polymers, e.g., low molecular weight (less than 600) polyethylene glycols and low molecular weight (less than 600) polypropylene glycols), glycerol, long chain PEG 8000 (about 180 ethylene monomers), methyl propanediol, methyl propylene glycol, neopentyl glycol, octylphenol-polyethylene glycol ether, PEG-4 through PEG-100 and PPG-9 through PPG-34, pentylene glycol, polyethylene glycol 200 (PEG 200 about 4 ethylene monomers), polyethylene glycols, polyglycol, polyoxyethylene cethyl ether, and octyl-polyethylene glycol ether, polyoxyethylene cetyl ether, polyoxyethylene lauryl ether, polypropylene glycols, tetraethylene glycol, triethylene glycol, trimethylpropanediol, tripropylene glycol.

The glycol compounds of this invention may yet further comprise ethylene glycol, propylene glycol, polyethylene glycols, polypropylene glycols, polyglycol and glycol derivatives including polyoxyethylene lauryl ether, octylphenol-polyethylene glycol ether, and polyoxyethylene cetyl ether. The preferred organic solvents of this invention are polyethylene glycols and glycols derivatives. The most preferred solvents are polyethylene glycols.

Polyalkylene glycols comprise polyethylene glycol (PEG) and polypropylene glycol. PEGs are generally commercially available diols having a molecular weight of from 200 to 10,000 daltons, more preferably about 200-300 daltons. Suitable PEGs can be obtained from Spectrum Laboratory Products, Inc, (Gardena, Calif., Molecular weight 200, Cat. #PO 107). The molecular weight of the polyethylene glycol (PEG) can range from about 200 to about 10,000. Generally, the polyalkylene concentration will depend on the polyalkylene used. Depending on the weight range of polyethylene glycol used, the concentration can be adjusted. The PEG at a concentration from about 0.1% to about 100% and PPG, when added to a PCR mix, have been shown to inhibit the effect of impurities on PCR.

Surprisingly and unexpectedly, the inventors have discovered that addition of DNAzoI Direct (Molecular Research Center, Inc.; Cat. # DN 131) to the reaction mix (see Example 1) resulted in an accelerated and complete digestion of a genomic DNA present at an ultra-low concentration (300 pg or less). In a typical reaction described herein, acceleration of a complete restriction digestion with a methylation-sensitive restriction enzyme Hin6I was achieved with a genomic DNA sample at a concentration of 2.33 ng/ml.

Efficient Amplification of Genomic DNA Using phi29 Pol in the Presence of the E. coli ssb Protein

Several useful methods have been developed that permit amplification of nucleic acids. Most were designed around the amplification of selected DNA targets and/or probes, including the polymerase chain reaction (PCR), ligase chain reaction (LCR), self-sustained sequence replication (3SR), nucleic acid sequence based amplification (NASBA), strand displacement amplification (SDA), and amplification with Qβ replicase (Birkenmeyer and Mushahwar, J. Virological Methods, 35:117-126 (1991); Landegren, Trends Genetics, 9:199-202 (1993)).

An exemplary method is known as primer extension preamplification (PEP). This technique uses random primers in combination with a thermostable DNA polymerase to replicate copies throughout the genome. Exemplary conditions that can be used for PEP-PCR are described in Zhang et al., Proc. Natl. Acad. Sci. USA, 89:5847-51 (1992); Casas et al., Biotechniques 20:219-25 (1996); Snabes et al., Proc. Natl. Acad. Sci. USA, 91:6181-85 (1994,); or Barrett et al., Nucleic Acids Res., 23:3488-92 (1995).

Further amplification methods may include, but not limited to, isothermal strand displacement nucleic acid amplification as described in U.S. Pat. Nos. 6,214,587 or 5,043,272.

Other non-PCR-based methods that can be used in the invention include, for example, strand displacement amplification (SDA) which is described in Walker et al., Molecular Methods for Virus Detection, Academic Press, Inc., 1995; U.S. Pat. Nos. 5,455,166, and 5,130,238, and Walker et al., Nucl. Acids Res. 20:1691-96 (1992) or hyperbranched strand displacement amplification which is described in Lage et al., Genome Research 13:294-307 (2003).

Other methods may include, but not limited to, are Nicking Enzyme Amplification Reaction (NEAR) as described in http://www.envirologix.com/artman/publish/article_314.shtml; nucleic acid sequence-based amplification (NASBA) as described in http://www.premierbiosoft.com/tech_notes/NASBA.html; and Cross Priming Amplification as described in http://www.readcube.com/articles/10.1038/srep00246?locale=en.

The use of the following polymerases has been previously described: Bst and Klenow fragment (https://www.neb.com/applications/dna-amplification-and-per/isothermal-amplification; http://www.neb-online.de/isothermal_amp.pdf); RPA (http://alere-technologies.com/en/products/lab-solutions/isothermal-amplification.html); thermophilic Helicase-Dependent Amplification (tHDA) (http://www.biohelix.com/products/isoampiii_enzyme_mix.asp; en.wikipedia.org/wiki/Helicase-dependent_amplification).

Phi29 DNA polymerase, for example, has proved useful in several amplification methods, such as for example, but not limited to Multiple Displacement Amplification (MDA). MDA can be used to amplify linear DNA, especially genomic DNA.

It has been previously shown that inclusion of E. coli SSB in reaction mixtures comprising linear DNA molecules leads to a much increased yield of amplified DNA products (see e.g., U.S. Publication No.: 20110065151, PCT/EP2009/056235, published as WO2009141430 A1, Joneja et al., 2011; incorporated herein by reference in its entirety). Other E. coli SSB may include, but not limited to, ET SSB (NEW England Biolabs; Cat. No.: M0249S), RecA (New England Biolabs; Cat. No.: M0249S), T4 gene 32 protein (NEW England Biolabs; Cat. No.: M0300S), and Tth RecA (New England Biolabs; Cat. No.: M2402S).

“Efficient amplification” as described herein is defined as the ability to amplify 0.35 ng of DNA (one half of the 0.7 ng is used for the digestion with Hin6I, and one half—as control) to no less than 10 μg of product. Importantly, the DNA is generally severely fragmented, which makes the amplification reaction using phi29 polymerase very inefficient.

### Quantification of PCR Fragments Using TapMan Quantitative PCR

Quantification of methylated or unmethylated CpG sites within amplified PCR fragments was carried out using TaqMan probe-based real-time PCR method as previously described (see e.g., 2011 MethyLight PCR Handbook, Qiagen; Zeschnigk et al., 32(16) Nucleic Acids Research (2004)).

In general, TaqMan probe-based real-time PCR method allows the direct quantification of the degree of methylation in a sample by using the threshold cycle values (C_{T}) determined by qPCR. In general, the PCR reaction exploits the 5′ nuclease activity of a DNA polymerase to cleave a TaqMan probe during PCR. The TaqMan probe contains a reporter dye at the 5′ end of the probe and a quencher dye at the 3′ end of the probe. During the reaction, cleavage of the probe separates the reporter dye and the quencher dye, which results in increased fluorescence of the reporter. Accumulation of PCR products is detected directly by monitoring the increase in fluorescence of the reporter dye (see e.g., TaqMan Universal PCR Master Mix Protocol, Applied Biosystems).

Determination of the DNA Methylation Status and/or Probability of a Methylation Status

Determination of the DNA methylation status and/or probability of a methylation status within the recognition site of the methylation-sensitive or methylation-dependent restriction enzyme was based on the comparison of C_{T} points in the amplification plots of restriction enzyme-treated and control parts of the same sample using ΔCₜ method (including the scoring protocol) (see e.g., 2012 EpiTect Methyl II PCR Array Handbook, Qiagen).

### EXAMPLES

The Examples that follow are illustrative of specific embodiments of the invention, and various uses thereof. They are set forth for explanatory purposes only, and are not to be taken as limiting the invention.

### Example 1

### Discovery of Biomarkers for Colorectal Cancer

### Samples

Cancer-positive samples were obtained from patients with established colorectal cancer as determined by a pathologist after resection of the tumour. Cancer-negative, or control samples, were obtained from individuals undergoing screening colonoscopy that did not detect any abnormalities. All samples were obtained from Caucasian subjects, matched by age and sex.

### Genomic DNA Preparation

High-quality genomic DNA is a prerequisite for a successful digestion reaction. Therefore, sample handling and genomic DNA isolation procedures are crucial to the success of the experiment. Residual traces of proteins, salts, or other contaminants will either degrade the DNA or decrease the restriction enzyme activities necessary for optimal DNA digestion. Genomic DNA was isolated using DNeasy Blood and Tissue Kit (Qiagen). Genomic DNA samples were diluted or resuspended in DNase-free water, or alternatively, in DNase-free 10 mM Tris buffer pH 8.0 without EDTA. The measurement of concentration of genomic DNA and calculation of the genomic DNA amount isolated was done with a PicoGreen reagent as described by Life Technologies, Invitrogen; (cat. #P7581).

### Measurement of DNA Concentration

The measurement of concentration of genomic DNA and calculation of the genomic DNA amount isolated was done with a PicoGreen reagent as described by Life Technologies, Invitrogen; (cat. #P7581).

### Restriction Digestion Protocol

The complete restriction digest was carried out according to the following protocol.

### Reaction Mix:

| | |
|---|---|
| H₂O | 50% |
| DNAzol Direct (Molecular Research Center, Inc.; Cat. # | 35% |
| DN 131) | |
| 10^{x} Buffer Tango (Fermentas; Cat. # BY5) | 10% |
| Hin6I (Thermo Scientific; Cat. # ER0481 | 5% |

The reaction mix was pipetted up and down to gently, but thoroughly mix the components and the tubes containing the reaction mix were briefly centrifuged in a microcentrifuge. Incubation of the complete restriction digest was carried out for 210 min at 42° C. in a thermal cycler. The digested sample was used in the subsequent amplification reaction.

Amplification of Genomic DNA Using phi29 Polymerase in the Presence of the E. coli SSB Protein

Mix the digested samples thoroughly by vortexing before use. Centrifuge the samples briefly in a microcentrifuge and proceed to step 1 of the amplification reaction.

Amplification was done as described below.

| Component | 1 reaction |
|---|---|
| 10^{X} Buffer (NEBiolab, cat#: B0269S) | 5 μl |
| 1.3M Trehalose | 13 μl |
| 0.5 mM Random Primer (1.1 μg/ul) | 5 μl |
| 25 mM dNTPs | 10 μl |
| 10 mg/ml BSA | 1 μl |
| 1M DTT | 0.25 μl |
| H₂O | 16 μl |

85 μl of the Master Mix was added to a PCR tube, followed by addition of 10 μl of digested genomic DNA. The sample was slowly vortexed and spun down before incubation. The sample was subsequently incubated for 2 min at 95° C. in a thermal cycler. After incubation, the sample was kept on ice for 30 seconds.

4 μl phi29 DNA Polymerase (New England Biolabs; cat. #M0269L) and 1 μl E. coli SSB-protein (10-20 ng/ml, Epicenter Technologies, an Illumina company; cat. #SSB02200) were added to the sample. The sample was then briefly vortexed, kept on ice 5 min, and spun down in a microcentrifuge. The sample was subsequently incubated for 16 h at 30° C. in a thermal cycler.

### Quantification of PCR Fragments Using TaqMan Quantitative PCR

Quantification of methylated or unmethylated CpG sites within amplified PCR fragments was carried out using TaqMan probe-based real-time PCR method as previously described (see e.g., 2011 MethyLight PCR Handbook, Qiagen; TaqMan Universal PCR Master Mix Protocol, Applied Biosystems; Zeschnigk et al., 32(16) Nucleic Acids Research (2004)).

Determination of the DNA Methylation Status and/or Probability of a Methylation Status

After the cycling program was completed, the C_{T} values were determined according to the following protocol. C_{T} was calculated separately for control and test parts of the sample, and then the difference was calculated (ΔCt). ΔCt>8 was considered significant and indicates unmethylated fragment (value 0). 2<ΔCt<8 was considered undefined and the fragment is not scored. 0<ΔCt<2 was considered significant, and the fragment was scored as methylated (value 1).

Each potentially informative fragment determined in the Discovery phase by microarray analysis is tested via qPCR in >=30 samples for each group and the frequencies of unmethylated score and methylated score were recorded. Fragments with differences >0.75 were combined into the composite biomarker. The fragments with higher differences were preferentially selected in order to determine the minimal number of fragments and to bring the probability of error to less than 0.001%. For example, if each fragment has probability of error <0.25, so that 0.25*0.25*0.25*0.25*0.25*0.25=0.000244 or 0.02%, so six fragments are insufficient and three additional are added: 0.000244*0.25*0.25*0.25=0.000003815 or 0.00015%. Considering that some of the fragments may fail in the reaction, the actual number of components in the composite biomarker was no less than 12 with the cumulative error less than 0.00000006 or less than 0.000006%.

### Selection of Biomarkers

Selection of informative fragments for TaqMan probe-based real-time PCR method was done based on (a) the highest difference in R=Cy5/Cy3 ratio between test and control samples, which has been determined previously in microarray-based discovery experiments; (b) consistent difference between test and control samples; and confirmed by (c) Fisher's Exact test (see e.g., Handbook on Biological Statistics found at http://udel.edu/˜mcdonald/statfishers.html). At the end of the Discovery phase up to 48 fragments for confirmation by qPCR are selected.

The techniques described in the preceding paragraphs allowed the identification of the biomarkers of Table 1.

### Example 2

### Detection of Colorectal Cancer

As discussed above, detection of colorectal cancer is based on the detection of a biomarker comprising one or more DNA fragments (see Table 1) and determining the methylation status of the one or more DNA fragments. Specifically, the detection of colorectal cancer is based on the probabilities (P) of these DNA fragments being methylated or unmethylated in healthy subjects and the error rates (ρ) associated with probabilities (or probability of errors) for each DNA fragment being either methylated or unmethylated in healthy subjects and subjects diagnosed with colorectal cancer (see Table 6).

First, the methylation status for twelve DNA fragments comprising the biomarker (see Table 1) in the sample from a subject was determined. Second, probabilities (P) of these DNA fragments being methylated or unmethylated and cumulative probabilities of error were determined. The cumulative probabilities of error for each DNA fragment were multiplied as discussed above to yield cumulative probabilities of error for healthy and diseased state (see Table 6).

The cumulative probabilities of error for healthy and diseased state for twelve DNA fragments comprising the biomarker were compared to each other. For sample 1, the cumulative probabilities of error for healthy state were less than cumulative probabilities of error for a diseased state. Thus, sample 1 came from a subject that did not have early stages (I and II) of colorectal cancer. For sample 2, the cumulative probabilities of error for healthy state were greater than cumulative probabilities of error for a diseased state. Thus, sample 2 came from a subject that had early stages (I and II) of colorectal cancer.

TABLE 6
Detection of Colorectal Cancer.

| | | Fragments | | | | | | | | | | | | Probability | |
|---|---|---|---|---|---|---|---|---|---|---|---|---|---|---|---|
| | | 1 | 2 | 3 | 4 | 5 | 6 | 7 | 8 | 9 | 10 | 11 | 12 | of error | Conclusion |
| Example 1 | Status | M | UM | M | UM | UM | UM | M | UM | UM | M | M | M | | |
| | Errors Health | 0.8 | 0.3 | 0.7 | 0.1 | 0.3 | 0.2 | 0.3 | 0.8 | 0.7 | 0.2 | 0.2 | 0.2 | 0.000001355 | Healthy |
| | Errors Cancer | 0.3 | 0.7 | 0.2 | 0.7 | 0.7 | 0.8 | 0.8 | 0.3 | 0.3 | 0.7 | 0.8 | 0.8 | 0.000531063 | |
| Example 2 | Status | M | UM | M | M | M | UM | UM | M | UM | UM | UM | M | | |
| | Errors Health | 0.8 | 0.3 | 0.7 | 0.9 | 0.7 | 0.2 | 0.7 | 0.2 | 0.7 | 0.8 | 0.8 | 0.2 | 0.000265531 | |
| | Errors Cancer | 0.3 | 0.7 | 0.2 | 0.3 | 0.3 | 0.8 | 0.2 | 0.7 | 0.3 | 0.3 | 0.2 | 0.8 | 0.000006096 | Cancer |

### REFERENCES

U.S. Publication No. 2012/038930
U.S. Pat. No. 7,727,718
U.S. Pat. No. 5,945,515
U.S. Pat. No. 5,001,050
U.S. Pat. No. 4,683,202
U.S. Publication No. 2006/0134650
U.S. Pat. No. 6,214,587
U.S. Pat. No. 5,043,272
U.S. Pat. No. 5,455,166
U.S. Pat. No. 5,130,238
Walker et al., Molecular Methods for Virus Detection, Academic Press, Inc., 1995.

TABLE 7

Gene information and Sequences.
KCNN4; chromosome location 19q13.2
(Ca-activated K-channel, regulates calcium influx)
SEQ ID NO: 01
GCGGCATCGGGTTACACAGTATCTAGCTGGCAACCAGGATCTAGTTCCAATTCCCTGCTTGGA
ATTATTTTCCAGAGCAGTTCCAAATCATCCCCTTCCTAGGATCACAAAAAGCACCTACCTACA
GTGCATTCCGTGCTAATTGGGAAAATATGTCTCCTTCCTCCAAGGCAGAGGCAACCCTTTAGG
CAGGTCCCAGAGATAGGTTCGGAGACCGAACAGATGGCCTGTAAACCTGAGGCAGAGGTCAGG
CAGCCGGAAGGGAGGGGCTTTCTAGGGTCTGTGTGTGCGTTTGGGGAGACTGAAGGCTGCAGG
TGGAGGATTGGCTGGGGGCTTGTCTGTTGGTTCCTCTCACCCCAGTTGATGGGAGTGTGGGCA
AATTTCAGCCAGCAAGAGGAGAAGGGGTCAAAGTGTGAACTTTCTCCACTGCTTGGTCCTAGG
GGGCCTCAACCTGCACCGCGGCACAGGACGGCCGCCGTGGCTGTCCGGGGTTCCCCCCTGCGC
ATTTATGCCTCCATCACCCTCACCTCTCGGCCACGGACAGCACCCAGGCGGTGGTCAGCCAGA
GGCCAAGCGTGAGGCCGAGCAGCAGGCGGCCAGGGTGCGTGTTCATGTAAAGCTTGGCCACGA
ACCAGTGGCGGAAGCGGACTTGATTGAGAG
ACER3; chromosome location 11q13.5
(alkaline ceramidase 3, positively regulates cell proliferation)
SEQ ID NO: 02
GCCTGGGCGGCGGCGGCGGCGGCGTGATGGCTCCGGCCGCGGACCGAGAGGGCTACTGGGGCC
CCACGACCTCCACGCTGGACTGGTGCGAGGAGAACTACTCCGTGACCTGGTACATCGCCGAGT
TCTGTGAGTGTGGCCTGAGGAGGGGAGTGGGGGCGAGAGGGCACCGGGCTGAGGAGACGCCGT
GTGAGGAAGGCAAAGAGCGAACCTGGCCGCGAAGGGAGGTGCCAGGCCTGGCCCCGGGAGCTG
GAATGCGGCGCCCTGGGCCAGCGGGAGGCTGAGAGGAGCGGGCCGGGAGTCCAGTGTGTAGAG
GGAGGAGTACCGGGGTCTGGGAGGGAGGAAGGGGGCCTGAGGATTGGGGGGGCAGAAGAGCAG
TGGGAAGTGGGGAGCCCCTGCTGGACCTAAGGGGGAAAGCCTGAAGAGCCGGGTTGGGAATGG
GAATTCCTGCCCGAGAGCGGAGTGGGGCCAGGCTGGGAGAGTGGAGGACCCTGCCCCTTGGAA
TGAGGGCCCAGGACACCTGCTCTGCTGTTGCCACCACCAGAAGGGTACAGTTCCTAGCTTCGT
CTTTCCCCCAATCCGTGAGAATTCTACCGTCTTTCCCTTCCCTTTTCACTGGAATATTAGACC
TCCTTGCTCACCTCCAGGGAACAGTTTCACTAGTCTGAGATCTGAACCATCCCACCCCTATCC
CCCAGGATGTCTTCAAGTACCAGAGGTCATCTGCTCTCTGAGTATGATTATTCAACTGTCATC
TTGCACCAGGAGTCGAAGGCATCTTGCACCTAGCCTGTACCTTCTGCCCCTGCCAGGCTCCCA
AGAGCACAGAGGACCAAGTCCCTGCTCCATTCTGTCCTATCCAACTATCTAGGAGTTAGGGGT
CATCTGAGGACACTACTTCCACCGACTGCACCTTCTGAGGATTTAAGCATTCTTCTTTAGCGG
CTGCTCTGTCAGGCACTGCTGGTCAGGTTGGGCTTGTTCTGTGTGCCTATGTGGGTGTCTGTC
GLI4; chromosome location 8q24.3
(GLI family zinc finger 4; glioma-assoc. oncogene family)
SEQ ID NO: 03
GCCTGCGGCAAGGCCTTCGGCCAGAGCTCCCAGCTCATCCAGCACCAGCGGGTGCACTACCGC
GAGTAGCCGGGCGGGGGCTCGGGGCTCGGCCTCCTCCCTGCCCCCAACCCACCCTCCACCCCG
TCCCCCACGGTGGGCACTGCCCAGCACCGCATGCCACGTGTCCGGAATAAATTCTTTTTGATT
GTTGGAAGTGGGAGCCGGCACCTGCCTGGGTGAGACCTTGGGGCAGCTTCCTATCCCCGAGGA
CCCGCTGCGGGATGGGGGTGATGGGGCTGCTCCACCAAGACCTGCCATACAGGGCCACGGGGT
CCCTGGGGTCTGGCGGGCGGCCCGAGTGTCGTAGGGGAGGATCTGAGGCCTGGAGGTGTCCTG
ACTTGCCCAAAGCTGATACCCCACCATCAACACGGGAGGCGGGGGGGGGCGCGCCCAGAGCAG
GGGTCGAGGACGGGGCCAGTCTAGAAGTGCTCACAGGCCTGGCCAGGCTGCCTGTCTGCCACC
TGGGTGAGGGGTCTCTGGCAACTCGGTTCCCTTATGTATTTGGGAGGCCTCTGCTTCTGTAAA
TGCAGCAGGCTTCCCCACGTGCCCTGTCAGCTCTGCTGCCTCCATTCAGTGGGGGGCCTGCTG
GGCAGCAGTGGCCCGGGCTTCCTCTGCACCAGCCCCTTGCCCTGGGGTGTGGGGGCCCAGGGT
GTTCAGGTCTTGACAGGTGTGGGCTGGTACGGCTGGGCCTGCCGGGCCCTCTTCAGAGCTGCC
GGGACACTGCTTCTGGGCAGGGGAGTCTGGGCCACGAAGCTCTGGGAGAGCTCAGCTGGGGGT
GGCTCCAAGTGCTGAGTGCCAGTGATTCTGCCAGTGCCTTCTCCCTGCCCTGCCTGTGCCCTC
CGGGACAGC
ZNF629; chromosome location 16p11.2
SEQ ID NO: 04
GCCTTCCCCTAGGCCAATTCTATAATCAGGAAAGAGAAAGGGCTTTTCGTTGCCGTGGGTGAG
CTGATGCTGGAGGAGCACAGAGCGATCCAGGAAGCTCTCTCCGCAGTGGGAGCAGATGTAGGT
CTTGGAGGACAGCAGCCCCCGCCTCTGGCTGAAGCCCTCCTGACCCTCCGGCGGCTTAAGGGG
CTGTCCGGGGGCCTCCGCTCTGCCCTCCGCAGCCCCGGGGTAGGAATTCCCTCTGAAAGGGAG
CCTTGGGGATCGTAACTGAGGAGGTTTGGGGGCTGCGTGTGCGATGAGGCCGTCTGCATTTTT
GTAGGGGTTTTCTCCGATGTGGATCCTCTGATGTTGCATGAAGATGCCCTCGTCGTTGAAGCC
CTTTCCGCACACGAGACACTTGTGCGGCTTGGCTCCCGGCGGCGGGGTCAGCAGGGAGGGGTC
CCCGAGCCCCAGCAGGCTGTCGCCCTGGGCCCTACGCGCTGGGGTCTTCCCCCTCTCATGGAT
CACCCGGTGCTGCTCCAGCTCGTGGGCTTCCAGGAAGGCCTTCCCGCAGTCGGAGCACACGAA
CAGGTTCTCGTCCATGTGCGTGCGGACGTGGGTAATAAGGTTGGAGCTCTGGCTGAAGCTCTT
GCCGCACTCGGGGCACTTGTAGGGCTTCTCGCCGGTGTGCGTGCGGCGGTGCTGGATAAGGTG
GGAGCTGCGGATGAAGCTCTTGCCGCAGTCGGAACACTTGTAGGGCTTCTCACCGGTGTGGAT
GC
MUC2; chromosome location 11p15.5
(Mucin 2; loss of expression—recurrence)
SEQ ID NO: 05
GCCTGCACCGCCAAGGGCGTCATGCTGTGGGGCTGGCGGGAGCATGTCTGCAGTGAGTGCCGT
CCCCGTGGGCTGCATCCTGGGGATGGGGTCCGGGCTTTGAGCTCCTGGGACGGGGCTGGGGGC
CCTGAGCACGGGTGGTCCAGGGAGAGGGGTCGGCCCCCTGCAGCCACGGACCAGGCTCCAGCT
TCGTCAGCCGGTGGTAGCAGGAAACCAGCAACTCCTATAGCAAGGGGCGGCCACGTAGCAGGG
GCAGAACCTGGGGTGGGCCTGGAGCTGTGGCGGCCGAGTGTGGGAGTGGGTCCCAGAGTGTGC
ACTCCCTGGCCCCCTGGCCACCCTGGGGATGGGAGCTGGGCGTCTGGCTCTTCCCGTCCCTCA
CACCACCCCGTGGTCCTCTGCAGACAAGGATGTGGGCTCCTGCCCCAACTCGCAGGTCTTCCT
GTACAACCTGACCACCTGCCAGCAGACCTGCCGCTCCCTCTCCGAGGCCGACAGCCACTGTCT
CGAGGGCTTTGCGCCTGTGGACGGCTGCGGCTGCCCTGACCACACCTTCCTGGACGAGAAGGG
CCGCTGCGTACCCCTGGCCAAGTGCTCCTGTTACCACCGCGGTCTCTACCTGGAGGCGGGGGA
TGTGGTCGTCAGGCAGGAAGAACGATGGTGGGTACCTGCTCGGGGGTCAGGTGTGGCGTGGGG
GCGGGGGAGCTCCTTCTGAACCTGCCCCAAGCGGAGACCTGGGAGTCTCTACCTGGGGAAGCT
GAGACACCCAAGGCTGAGGGGTGCCTGGGGTGGGGGGCGCTGAGAGGCATCAGGCTCACATCT
GCGGGGAAGCTGCGGGCTGTCTGTGGCCGTCCTGCATGGGCCCCGCTCATCCCTGGCCTTTTC
CACAGTGTGTGCCGGGATGGGCGGCTGCACTGTAGGCAGATCCGGCTGATCGGCCAGAGTAAG
TGGCACTGCCCCGGCCACCCCTCCCCAGCCACCCCTCCCTGCCTGCCCTGGCCACCCTCCCCG
GCCACCCCTCCCGGGCCTGCCTGAGACCCCCAGCTTCAGCTGGAGCTGAGGTGGCCCCTCCGT
CCCACAGGCTGCACGGCCCCAAAGATCCACATGGACTGCAGCAACCTGACTGCACTGGCCACC
TCGAAGCCCCGAGCCCTCAGCTGCCAGACGCTGGCCGCCGGCTATGTGCGTGTTGGGGGC
HDAC4; chromosome location 2q37.3
(Histone deacetylase 4 promotes CRC via repression of p21)
SEQ ID NO: 06
GCAATCATAGCTCACTGTAAGCTTGAGCTCCTGGGCTCAAGTGATCCTCCTACCTCAGACTCC
CAAATAGATGGCAGTTAATTAAAAAAACAAAATTGTAGAGAAGGGGTCTTGCTATGTTGCCCA
GGCTGGTCTCGAACTCCTGGGCTCAAGCCATTCTCCCACCTCAGCCTCCTGAGTAGCTGGGAC
TACAGGTGCACACCACTGCACCCAGATACGTTTTCTTCTTTTTTGATGAAACAAGATCTTGCT
CTGTTGCTGGGGCTGGTCTCAAACCCCTGGGCTCACGTGATCCTCCCGCCTTAGCTTCCTAAA
GCTCTGGGATTACGAGCGTGAGCTGCCTCACCCGGCCACTGGTGGGTTGCTTTTTGTTGGTCT
TGCTCCCCTTATGGAGGAAGAGGGGACGGTGAGAGGGTACGGGATAAGCAGGCATCCTGGCAA
CCAGAGTGGCCCGAGGAACTTTCTGTGGAGGAAATTTAGTGAATCAGGGGCTCCGGGCTGGCT
CCAGAGTGGGGCTTCCACCAGCTGGTGATTCTTCCTGGAGGATGAGGCTCAGGCCAGGGAAAG
GATGAGCAAAGCATAGAGTGGGGTGTGTGTGCGAGGCAGCCACCGGATGCCCGAGGCATAGAG
TGGGGAGTGCGTGCGAGGCAGCCACCAGACGCCCGAGGCATAGAGTGGGGTGTGTGTGCGAGG
CAGCCACCGGACGCCTGAGGCATAGAGTGGGGTGTGCGTGCGAGGCAGCCACCGGACGCCCGA
GGCATAGAGTGGGGAGTGCGTGCGAGGCAGCCACCGGACGCCTGAGGCATAGAGTGGGGTGTG
CGTGCGAGGCAGCCACTGGATGCCTGTGCTCCATGAGTGGCTGCGCTGGCACAGCAGGACTGG
CGCCCATGGGATGCCACCCACGTCACACTGTCGTCCCTGTGTATTCTTCAATCCCTCTACGAC
AGGGGTCCCCACCTCCGGCCGTGGACGGGTAGCAGCAGTCCCTGGCCTGTTAGGAAATGGGCC
ACAAAGCAGGAGGTAAGTGGCAGGCTAGGGAGCATTCCCGCCAGAGCTCAAGCTCCTGCCGGA
TCAGTGGTGGCATTAGATTCTCACAGGAGTGTGATCCTGTTGTGAACTGTGCGTGTGGGGGAT
TTAGGATGCATGGTCCTTATGAGAATCTAACGCCTGATGATCTGAGGTGGTGGAAGTTTCATC
CCGAAACCATTCTCCTGCGTCCCCCACCCCTGTCCACAGAAAAAACCATCTTCCACGAAACCG
GTCCCTGGTGCCAAAAAGGTCGGGACCGATTGCCGCTCTACAACAAATGC
PLIN3; chromosome location 19p13.3
(Perilipin3 binds directly to the GTPase RAB9 (RAB9A))
SEQ ID NO: 07
GCCCTCTGGTGGCTGCTGTGGGGAGGAGACTGTGGTGGATGAGGGCGGGAGCTGGTGAGCAGG
ACAGAGGGGACTGCGTTAGTGATGAGATTCCAAGATGCCCGGGAGAAGTGGCAGGGACGAGGC
GGCAGTGAGTGTCGGCACAGACCCCAGGAGGCCGACAGCGGCTTCCGGTCAGGGGGCCTGGGG
AGGGGTCCCAGAGCAGCCCGCTGGCCACACTTACCCAGTTCGGCATCCGTAAGGGGCAGGTGG
TTGTCCGCCCACTCCTCCGACTTCCCCAGCACCGTGTCGACCCCACTCAACACCATCTGGCCC
AAGCGGGAGCCCATGACCGATTGGACGCCGCCGGTCACTACGGACTTTGTCTTGTCCACGCCG
CTCTGCACAGCACCGCGGGTCGCGTCCACCGCCTCCGACAATTGGGTGGCCACCGTGTCCTTG
GCGCTAGACACCATCTCTTGGGCCCCCGACACCTTAGACGACACAAGCTCCTTGGTGTCCGCC
AGGACCTAGGAGATGCAACAGCATCAGCATCTCTGCCTTCCCTCCATATCTGGGCACCCCTCC
CCTGCACCCCAACTTCCAGGGAGACCGAGGCGGGGAGC
ZNF30; chromosome location 19q13.11
SEQ ID NO: 08
GCCGGGCATGCTCGGCGGTGTGACGGCTCAGGACTGCATTTCCCAGAGGCTGCAGCTATCCGG
CCAATGTAGCCTGAAACTACATTTCTCAGCGGCCACTGGAACGACCTCAATCTCTGCCTCCTC
GCCAGTTCATTGTGGTCGTTGACCCGGCAGCGAGCTTTGGAGTTCATCGAGGGAGAAGTCAGC
GCCCAGCTCCGAGGTTGGAGCAGCCCCGCCGGGCAACTTGAATTTCTGCAAACGAACACAGCA
CCGGGAGCTCTGCAGACCTGTGTCGGCGCGGAACCCGGACTGAGACATGCGTGAGCGTTGGGT
GGACCGGGCGAGGATCCCGGGCCGGCGAGTGCGGGAGCGGCAGGGCAGGGAGGGTGCGTCGGC
CGGGGCCGGTGTGCATCCGCGAAGACTGGGTGCATGGCCTCCATGCGAACCTGAGCTATTAAT
ATTTGTTACTATTTTGGATAAAATCACTGTAATTGATTTATGTAAAGGAGCAAAAGACTCTTC
AACTCTCAGTTTAAAAAGGAAACGATAGTTATGATACCTTTTGCATGCAGCGGGAAGAAATGG
GATTGCCAGGAAGCCTCTTCTTGTTTGGAAAAAACTGTATAAAGTATTTACACCTTTTAAAGA
TGAGAGCAATGTCATCTGAAAATTATCAGTGCAGGGAAAAGGACTTCAAAGGATCTGTTGTGC
AGATTACTTAACTAATGACAAAATTATGTAAGAAAGGAGAGCAAGATGACAGCTGTAAACATT
TCCATCAATCTCCATATTGCACAGAAATAGGACCCAGCTTTTTCTTAAGGTTCTTCAATTTTG
CATTATCCCACAGCAGTAGCTCTCTCTCTCTAGCTGCTAGGGGACAGAGGAAATTGAAATGTC
AGAGAATCTTTTCTGTTGGTTTTTTATTTGTTTGTTTTTAAAGAAGAGTTGCTCTTAATTTTT
TAGTTAGAATTAAAAGAAAGCATGCCAGAGAAACTTACGTTTTAAGTAAAAAGTGGAAACAGG
TCGGGTGCCATGGCTCATGCCTGTAATCCCAGCACTTTGGGAAGCTGAGGCGGGTGGATTGCC
TGAGCTCAGGAGTTCGAGACCACCAACATGGCAACATGGTGAAACCCCGTCTCTACTAAAAAT
ATAAAAATTAGCCAGGCATGGTGGCGTGC
CELSR1; chromosome location 22q13.3
(cadherin, EGF LAG seven-pass G-type receptor 1)
SEQ ID NO: 09
GCTGGGTGCGAATCACACCGGACGTGGGCTCGATGTAGAAGTCCCCATCGCCGTCGTCCCCAC
CCTGGAAGGTGTACAGCAGACGCCCATTGGGACCTGAGTCCCGGTCCGTGGCAGAGACCTGGA
GGATGCTGGTCGAGGGTGGAGCATCCTCAAAGATGGAACCCTGGTAGAAATCCCACAGGAACT
GGGGTGCATTGTCATTGGCATCGAGGATGAGGATCTCTAGGGTGGTGGTGTCTGATTTCTGCG
GGATGCCGTTGTCCTGGGCCATGATGGTCAGCGTGTAGGCGACCTGGTTCTCATAGTCCAGCT
CCATCATGGTGTACATGGTGCCACTGTCGGGGTCAATGCGGAACTGCGGCACGGGGTCCTGAA
TCACGTAGGTGATGCGGGCATTCTCTCCTGTGTCCTCATCGTTGGCACTGAGGGTAGCAATGG
AGGTGCCCACAGGCCTGTCCTCACTGACACTCACTGTGTAATGGGAGCTCTGAAAGACAGGCC
TGTGGGTGTTGGCATCAGTGACGTTGATTAGGACATGCGCAGTGTGCGACCGTGTGCCGTCGG
ATGCTGTCACCGCCAGCACGTACTGCTGCTCCTGCTTGTAGTCCAGAGGTAGCGCCAGGGTGA
TGAGGCCGCCCCCTCTCTGGCTGCTGAGTGCAAAGCGGTTCCGGGTGTTGCCGCCTGTGAGCT
GGTAGGTAATCACACTGTTGGCGTCACGGTCGCGGGCCTGCAGGGTCAGCACGCTGCTCCCCA
CGGCCGCATCCTCATTCAGACGAAGCTCGTAGGTGGGCTGCGTGAACACCGGGTCGTTGTCAT
TCACGTCCAGCACCGTGATGGACACGCTGGTGGAGGAGCTCATGGGGGGCCAGCCGTGGTCCA
CCGCCTCCACCCCGAAGCTGTAGTGCTCCACCTCCTCGCGGTCCAGCTCGGCACACACTGTGA
TCCAACCGGAGCTGTTGTGGATCTGGAAGGGGAAGTCAGGGGTGGGGGCAGGATTCTTAGGCC
CAGC
unknown; chromosome location chr8: 1094666-1094715
SEQ ID NO: 10
GCGTTTTCACCGCCCTGTGCTGGAAAGGCACTTAGGAAGATAATGAATATAAACTCACACTAT
CTGGACACAGATGGAGAAGGCGGTGGAGCATTCGAGTGGATGATTAAAGAGAAAAACAAATCA
GGAGGTAAAATTACTGTTTATGGGCCAGGGAGGCCACGTCCTAAAGTTTAGTGGAATTGTGCT
TTAGAAAGAATGCTGTAAGAAATCCAGAAGCTGTGAAGACGGTAAAGACAATGATGACAGTGA
GCTTTCTTGTTTCTTTGAGGCTTCCGAATGCTCCTCCCCAGTCTGCGTCCTGCTTTGACTGGA
CGTTGCAAACAAAAGATTCTTGCTTTGTCTGTCTCCATCCTTTCGACCACCTCCAGAAGCTAC
AGGAAATAAACGCTCTTTCCATCCTGGTCCCTTTGCCACCCACAAATACAGAGAAGTTGCGTC
TAGGTAAATATTAATCTCTGCTTCTGCTTTTCCTTCCTGTGTGCTGTGAATACAGGCCCTGTC
TGCAGTTTTACTTTTGGCTGAAGTAGCCCATGCTCTAGGGTCCATCCAGGAAACACACAGCGC
ACAGTCAAACCGCAGACGGCCTGTACCCACAGTCAAACCACAGACGGCCTGTATGCACAACCA
AACCGCAGACGGCCTGTACCCACAGTCAGACCGCAGACGGCCTGTATGAACAGTCAAACCACA
GACGGCCTGTACCCACAGTCAAACCGCAGACGGCCTGTACCCACAGTCAAACCGCAGACGGCC
TGTACCCACAGTCAGACCGCAGATGGCCTGTATGAACAGACAAACCACAAATGGCCTGTATTC
ACAATGCAAAGGAAGGAAAAGCAAAAGCAAAAGTTAATATTCACCTAGATGCAACTTCTCTGT
ATTAGTAGGTGGCCAAGGGACCAGGATGAAAAGAGCATTTATTTCCTGTAGCTTCTGGAGGTG
GCCAGGAGGATGGAGACAGACAAAGCAACCAGAAACACAACTTTCCCCCCAGCCACCATCCAG
AAACACAGCTTCCCTCCAGCCACTGTACAGAAACACAGCTTGCCCCACCCAGCCACCATCCGG
AAACACAACTCCCACCCACCCACCATCCCTCCAGGAAGCCGCTGTTTTTAATCCCCTCCCATG
AGTTATGAATTGTGTCTGGTGTGGTGGACCCTGGAGCATGGGCTTGTTGGCTGCGGTTCCACT
CGCCCAGCGTGGGGCCTGGGAGACCTGGCTGAGCTGGTGTGTGGTGTCCTCTGTACATGACTC
CACTGTGGTCTCCCGTCCTGTGGGTGTGCATGCTTCATCCATCCATTGCAACGTCAACAGACC
CCTCTCCTCCTTCCACTTCTCTCCTCCTGTTTTCTAGTTTGAAACTCTTACCAATAATGCTGC
TGTAAACATCTTCTGCATATTTTTGGTGAATCTATGGATGTATTCTTTTTTTTATTATACTTT
AAGTTTTAGGGTACATGTGCACAATGTGCAGGTTAGTTACATATGTATACATGTGCCATGCTG
GTGTGCTGCACCCATTA
unknown; chromosome location chr2: 583162-583222
SEQ ID NO: 11
TAATAGTTAATGCTAGCAAACAGTGAAATGTAATTAGGGCAGAGAGACGCTGAGGCTCATTAG
AAAGAACAACAACGCTGAGCTGTGAGCCGGAGGAGGCAGCCGGGTTCTGATGGAAGCTGCCTC
GACCACCAGAACAACACCGCAAGCGTCCAGCAGCAGTAAGGGGCACAAGCTGCCTCGACCACC
AGGCCAATGCCACCAGCGTCCAGCAGCAGCGAGGGGCACAAGCTGCCTCGACCACCAGAACAA
TGCCGCCAGCGTCCAGCAGCAGTGAGGGGCACAAGCTGCCTCGACCACCAGAACAACGCCGCC
AGCGTCCAGCAGCAGTGAGGGGCACAAGCTGCCTCGACCACCAGAACAATGCCGCCAGCGTCC
AGCAGCAACGAGGGGCACAAGCTGCCTCGACCACCAGAACAACACCGCAAGCGTCCAGCAGCA
GCGAGGGGCACGGAGAGCAGGCAGTGCAGAAGTCAAACCCCTAACAGCCACAGGAAACTCAGG
GCAAACGGAAGCGTTTCCATTCTCCAGCCCTTCTTTCAATATTCTTAACATGAGCAATCCATG
AGCCCTCATTTTGCAGCCCACAGAACCTCAGCCAGCGTGTGAGGAAGAAGCTCCAGGCGGCGG
CAGCCAGCGTGTGAGGAAGAAGCTCCACGCGGCGGCCAGTGTGTGAGGAAGAAGCTCCACGCG
GCGGCGGCCAGTGTGTAAGAAAGAAGCTCCACGCAGCGGCCAGCGTGTGAGGAAGAAGCTCCA
CGCAGCGGCCAGCGTGTGAGGAAGAAGCTCCAGGCGGCGGCGGCCAGCGTGTGAGGAAGAAGC
TCCACGCGGCGGCCAGTGTGTGAGGAAGAAGCTCCACGCGGCGGCCAGTGTGTGAGAAAAGCT
CCACGCAGCGGCCAGCGTGTGAGGAAGAAGCTCCAGGCGGCGGCCGCCAGCGTGTGAGGAAGA
AGCTCCACGCGGCGCTTGCTCAGGGAATCCTGCTCCAGGGCGTGCTCACTTGCTGTTATTGTG
TTTTATTTTTCCTGAGACTGTAAATGGAGCGGATAGAAGTTCAGAACCATCGGTCCCTCTTCT
TCCTGGGTCATCCTGAGCTCGGCAGTGAGAGCACCTACGACTAGGGAGCGGCCGAGCAGAGGG
AACAAGGCCGTGCCCGCTAAGGTTCTCCCGGGACGGTGGCGAGCCCACGCTTGCCAGGCATGA
CGCCTCGACCTCCAGCGTCCAGAGCGTCCCTTCATTGGTTCACAGGAACTTTTCACATGTGTC
CGTCCACTTTTCTTAGGAATATTTATTTAGGTGAGGTTATTCATTCTGACACTGGAAGAAAAG
TGCAAAACCTCGTGTGGACTTCGTAGGTGGAGCATTTGAGTTATCATCGGAAAACTAGAGCCC
GGACTGTATGAGGAAGGTAATTCATGTTTACAACTGATTATTGCTTTGGGTGATTTTCTCTAA
TGCAATAATAAAAATAGTAGAAAGAAACTTTTCAACTGTGAAACCCAAACTTAATATTACTAT
ATCATTATTATCAGTCTTTAAACACCTATTTCAGACAAGTTTTTTAAAATATAAAGACAAGAC
CTAATAAGAGGTGTGAGTTTTACAAATATACCAGAAAAGTGTGTGCCTGAATAAGTGTTGACC
CCTCAGAGTGACCCCTGCTGGTCGCAGGGAACCTGTTCCCATCACGTCCCCACTCACCCACAA
GGCAGC
NIPAL3; chromosome location 1p36.12-p35.1
SEQ ID NO: 12
CGAAATGCCTGCCAACTTCTGACTGGCAGGCAGTCTGGCAAATCAAATCGCGACCTTTGAAAG
CAAAACACTGCAGCATCTTGGCAGCTCTGAATTGGGAAGGGATGAAGGAGGCTGTGCCTCCGG
GTTGCACGAAGAGTCCGAGTCATTTCTCAGAAGGTTTTGATAGGTGGGCCTTAGAGGAGACGC
CGCCGGTGAGTAGTGATTAACACCGGGAGGAAGGGGAATTGAATTTAACCTTCGTTTTTTTCT
GGAAAAAGCGAAGTCACCTAACGTCCCCTAGTGTACATACCCTTCCTTCTTACTGTCACCAGC
CTCGCCAACCTGGGTCCCGTTGCCTTGGAATGTTCTTTCCAGTTTTGCATCGAGGCCAAGAGG
AGCGGGGGCATGGGCTACCTTACTAAAGGTGATGCCAGGCTCTACCAAACCAGGAAGTGACAT
GGAGTTAACTTTGCCAGAATTTCTCCTCTTCGTGCCGAGCGGCTCGGGCTTCCTGGCGGCAGC
AGATGGTGGAGTTAGCAGGTGGGATGAGGGGAGGCGTTCTTGGTCTAAGCCCGCTTCTGGAAC
AGAGGTGCTGTCTCCTCGAGTTGTAAGTTTCCAGCTCAGTGGGACGGGACGGAAGAATGTAAC
CTTCTCGTGAGCCAAAGCCGAGGAACGGGAAGCTTGGCAGGGAACTGGCGCTCACCTCCAGAA
GCCAGATCGTCGGGTGGTGGGAAAGAGCGTGTTTTATTGATTTGTTCAGAAAGAGGCAAATTC
GAATACAGACGCTATGAGCCACGGCTGTCTCATTTGTAAAACGTGCTCTGTGGGATTGGTGAA
ATCCGTCATCGAGATAAACGGGGTGGGAATGGAAGCAGAGCACCTAGTGAATTCTCATTCCTT
CCTTGGGTCAGTGACCACGTGCTCTAATTGTGGGGTGGTTGACAACGCAGAGGTGACTGCTTG
CCTCTCGGGCATATGTAGGTCCTGAAGAAATGCTTCGAAATCAGGAAAAGAGAGTCACCAGGT
GAAAAGTATGTGTCTTATAAGGGAGTACAGCTTGCAAAGGGTTCCTCCAGGCTTTCAGTCCGG
ACTCCCACCCAGCTGAGGGAGAGCCTTCAATCTTTGCAGGCGATGCTTCAGAGGCCTGCGAGT
TCCTGAGGCAGAGAGGGAAGCTGCTTTTTAAAAGAAAAATTAAACCACAGCAATGCCAACCAC
CACAAACAAAAGCAAAACCAGAAAAGCACTTGGGCAAACTACTCTGAAGGATGTTAGGAGGCC
TGGGTTCCCACCACTCCCTTGATTGACATGTCGCTAAGGTCTGTTGGCTTTCTCTGACCTCCT
GTGGGTGGGGCTGAGTATATCTGCTTGTTGAAGCTCTGGAGTTGTGGTTGATTAGGCCTTAGA
AAGGCATTCTTGACTGCGAAGGGGCCACAGTGCACCCAGTGCTTAACCAGCCTGCATTTAGTC
AGTCGGTTGGTATGTATACAAGTCACTGTCAGGCTCTGGGCTAGATCTCATTTAGTCAGTTGG
TTGGTATGTATAGAAGTCACTGTCAGGCTCTGGGCTAGATCTCAGCTGGGAGCAACTGAACAG
GGTATTCCGCAAACACCTCACTGGAGTTTGC

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

What is claimed is:

1. A method for detecting cancer, comprising:
(a) analyzing a biological sample from a subject to detect a presence of a biomarker in the sample, comprising
i. obtaining a DNA sample from a subject;
ii. digesting the DNA sample with a methylation-sensitive restriction enzyme in the presence of a glycol compound;
iii. amplifying the digested sample of step (ii);
iv. quantifying amplification results from step (iii) using a real-time quantitative PCR; and
v. analyzing DNA methylation status within a recognition site of the methylation-sensitive restriction enzyme to detect a presence of the biomarker in the sample;
(b) determining a methylation status of the biomarker detected in step (v); and
(c) comparing the methylation status of the biomarker detected in the sample to cancer-positive and/or cancer-negative reference methylation status of the biomarker to detect whether the subject has cancer.

2. The method of claim 1, wherein the step of comparing further comprises:
(a) determining probabilities (P) of the biomarker being methylated or unmethylated;
(b) determining cumulative probabilities of error (ρ) associated with probabilities (P) of the biomarker being methylated or unmethylated;
(c) determining cumulative probabilities of error for the biomarker in a healthy and a diseased state; and
(d) detecting that the subject has cancer if the cumulative probabilities of error for the biomarker in the healthy state is more than the cumulative probabilities of error for the biomarker in the diseased state.

3. The method of claim 1, wherein the subject is a mammal, wherein the mammal is a human.

4. The method of claim 1, wherein the sample is a biological sample comprising blood, blood plasma, urine or saliva.

5. The method of claim 1, wherein the biomarker comprises one or more DNA fragments of SEQ ID Nos. 1-12.

6. The method of claim 1, wherein the cancer is colorectal cancer.

7. The method of claim 6, wherein the colorectal cancer comprises early stage I and 11 colorectal cancer or late stage colorectal cancer.

8. The method of claim 1, wherein the DNA comprises a genomic DNA.

9. The method of claim 1, wherein the DNA sample is between about 1 pg and about 1 ng.

10. The method of claim 9, wherein the DNA sample is about 300 pg.

11. The method of claim 1, wherein the methylation-sensitive restriction enzyme comprises Hin6I.

12. The method of claim 1, wherein amplifying comprises amplifying using phi29 DNA polymerase.

13. The method of claim 12, wherein amplifying further comprises amplifying using a single stranded DNA binding protein of E. coli.

14. The method of claim 1, wherein the real-time quantitative PCR comprises TaqMan qPCR.

15. The method of claim 1, wherein determining the DNA methylation status and/or probability of a methylation status comprises determining threshold cycle (CT) values.

16. A biomarker for detecting cancer, wherein the biomarker comprises one or more DNA fragments of SEQ ID Nos. 1-12.

17. The biomarker of claim 16, wherein the cancer is colorectal cancer.

18. The biomarker of claim 17, wherein the colorectal cancer comprises early stage I and II colorectal cancer or late stage colorectal cancer.
